(19) United States
(12) Patent Application Publication
(10) Pub. No.: US 2026/0102485 A1
(43) Pub. Date: Apr. 16, 2026

(54) NEUTRALIZING ANTI-TL1A MONOCLONAL ANTIBODIES

(71) Applicant: CEDARS-SINAI MEDICAL CENTER, Los Angeles, CA (US)

(72) Inventors: Janine BILSBOROUGH, Adelaide (AU); Stephan R. TARGAN, Santa Monica, CA (US); Bradley HENKLE, West Hollywood, CA (US)

(73) Assignee: CEDARS-SINAI MEDICAL CENTER, Los Angeles, CA (US)

(21) Appl. No.: 19/336,225

(22) Filed: Sep. 22, 2025

Related U.S. Application Data
(63) Continuation of application No. 18/191,712, filed on Mar. 28, 2023, now abandoned.
Application No. 18/191,712 is a continuation of application No. 17/102,189, filed on Nov. 23, 2020, now abandoned.
Application No. 17/102,189 is a continuation of application No. 16/384,521, filed on Apr. 15, 2019, now abandoned.
Application No. 16/384,521 is a continuation of application No. 15/792,266, filed on Oct. 24, 2017, now Pat. No. 10,322,174.
(60) Provisional application No. 62/413,188, filed on Oct. 26, 2016.

(51) Int. Cl. A61K 39/395; A61K 39/00; A61K 48/00; A61P 1/02; A61P 29/00; A61P 37/00; C07K 16/24; C07K 16/28

(52) U.S. Cl. CPC A61K 39/39533; A61K 48/005; A61P 1/02; A61P 29/00; A61P 37/00; C07K 16/241; C07K 16/2809; C07K 16/2875; A61K 39/3955; A61K 2039/505; C07K 2317/24; C07K 2317/33; C07K 2317/56; C07K 2317/565; C07K 2317/76; C07K 2317/90; C07K 2317/92

(57) ABSTRACT

Described herein are methods and pharmaceutical compositions for the treatment of inflammatory bowel disease (IBD), Crohn's Disease (CD), ulcerative colitis (UC) and medically refractive-ulcerative colitis (MR-UC). In particular, disclosed are anti-TLI A antibodies useful for the treatment of IBD.

### CROSS-REFERENCE

This application is a continuation of U.S. application Ser. No. 17/102,189, filed Nov. 23, 2020, which is a continuation of U.S. application Ser. No. 16/384,521 filed Apr. 15, 2019, which is a continuation of U.S. application Ser. No. 15/792,266 filed Oct. 24, 2017, now issued as U.S. Pat. No. 10,322,174 on Jun. 18, 2019, which claims priority to US Provisional Application 62/413,188 filed Oct. 26, 2016, the entirety of which is incorporated by reference herein.

### SEQUENCE LISTING

The instant application contains a Sequence Listing which has been submitted electronically in XML format and is hereby incorporated by reference in its entirety. Said copy, created on Mar. 27, 2023, is named 56884-728_303_SL.xml and is 67,992 bytes in size.

### BACKGROUND

Inflammatory bowel disease (IBD) refers to a collection of intestinal disorders causing inflammatory conditions in the gastrointestinal tract. The primary types of IBD are ulcerative colitis (UC) and Crohn's Disease (CD). These diseases are prevalent, with about 1.86 billion people diagnosed globally with UC, and about 1.3 million people diagnosed globally with CD. Unfortunately, there are a limited number of therapies available for IBD patients, and the development of new therapeutics has been hampered by sub-optimal results in clinical trials. Accordingly, there is a need for novel therapeutics to treat IBD.

### SUMMARY

The present disclosure provides antibodies useful for the treatment of IBD. In one aspect, provided is an antibody or antigen-binding fragment that specifically binds to a TL1A polypeptide. In some embodiments, the antibody or antigen-binding fragment comprises: a heavy chain comprising complementarity determining regions (CDRs) of SEQ ID NOs: 6-8 and a light chain comprising complementarity determining regions (CDRs) of SEQ ID NOs: 14-16. In some embodiments, the antibody or antigen-binding fragment comprises: a heavy chain comprising complementarity determining regions (CDRs) of SEQ ID NOs: 22-24 and a light chain comprising complementarity determining regions (CDRs) of SEQ ID NOs: 30-32. In some embodiments, the antibody or antigen-binding fragment is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. Further embodiments provide for pharmaceutical compositions comprising a therapeutically effective amount of the antibody or antigen-binding fragment, and a pharmaceutically acceptable carrier. Further embodiments provide for a method of treating inflammatory bowel disease in a subject in need thereof, comprising administering to the subject a therapeutically effective amount of the antibody or an antigen-binding fragment. In some embodiments, the inflammatory bowel disease comprises Crohn's Disease, ulcerative colitis, medically refractive-ulcerative colitis, or a combination thereof. In some embodiments, prior to administering the antibody or antigen-binding fragment to the subject, the subject over-expresses TL1A. In some embodiments, the subject comprises a risk variant associated with the inflammatory bowel disease.

In another aspect, provided herein is a polypeptide comprising: one or more complementarity determining regions selected from the group consisting of SEQ ID NO: 6, SEQ ID NO: 7, SEQ ID NO: 8, SEQ ID NO: 14, SEQ ID NO: 15, SEQ ID NO: 16, SEQ ID NO: 22, SEQ ID NO: 23, SEQ ID NO: 24, SEQ ID NO: 30, SEQ ID NO: 31, and SEQ ID NO: 32.

In another aspect, provided herein is an antibody or antigen binding fragment that binds to the same region of human TL1A as a reference antibody comprising the heavy chain complementarity determining regions (CDRs) of SEQ ID NOs: 6-8 and the light chain complementarity determining regions (CDRs) of SEQ ID NOs: 14-16. In some embodiments, the reference antibody comprises a heavy chain variable domain of SEQ ID NO: 5 and a light chain variable domain of SEQ ID NO: 13. In some embodiments, the antibody or antigen-binding fragment is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. Further embodiments provide for a pharmaceutical composition comprising a therapeutically effective amount of the antibody or antigen-binding fragment, and a pharmaceutically acceptable carrier. Further embodiments provide for a method of treating inflammatory bowel disease in a subject in need thereof, comprising administering to the subject a therapeutically effective amount of the antibody or an antigen-binding fragment. In some embodiments, the inflammatory bowel disease comprises Crohn's Disease, ulcerative colitis, medically refractive-ulcerative colitis, or a combination thereof. In some embodiments, prior to administering the antibody or antigen-binding fragment to the subject, the subject over-expresses TL1A. In some embodiments, the subject comprises a risk variant associated with the inflammatory bowel disease.

In another aspect, provided herein is an antibody or antigen binding fragment that binds to the same region of human TL1A as a reference antibody comprising the heavy chain complementarity determining regions (CDRs) of SEQ ID NOs: 22-24 and the light chain complementarity determining regions (CDRs) of SEQ ID NOs: 30-32. In some embodiments, the reference antibody comprises a heavy chain variable domain of SEQ ID NO: 21 and a light chain variable domain of SEQ ID NO: 29. In some embodiments, the antibody or antigen-binding fragment is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. Further embodiments provide for a pharmaceutical composition comprising a therapeutically effective amount of the antibody or antigen-binding fragment, and a pharmaceutically acceptable carrier. Further embodiments provide for a method of treating inflammatory bowel disease in a subject in need thereof, comprising administering to the subject a therapeutically effective amount of the antibody or an antigen-binding fragment. In some embodiments, the inflammatory bowel disease comprises Crohn's Disease, ulcerative colitis, medically refractive-ulcerative colitis, or a combination thereof. In some embodiments, prior to administering the antibody or antigen-binding fragment to the subject, the subject over-expresses TL1A. In some embodiments, the subject comprises a risk variant associated with the inflammatory bowel disease.

In another aspect, provided herein is a composition comprising a peptide having SEQ ID NO: 7. In some embodiments, the composition further comprises one or more peptides selected from SEQ ID NOs: 6, 8, and 14-16. Further embodiments provide for a method of treating a subject having an inflammatory bowel disease, the method comprising administering to the subject an effective amount of the composition.

In another aspect, provided herein is a composition comprising a peptide having SEQ ID NO: 23. In some embodiments, the composition further comprises one or more peptides selected from SEQ ID NOs: 22, 24 and 30-32. Further embodiments provide for a method of treating a subject having an inflammatory bowel disease, the method comprising administering to the subject an effective amount of the composition.

In another aspect, provided herein is a method of treating an inflammatory bowel disease in a subject in need thereof, the method comprising administering to the subject an effective amount of an anti-TL1A antibody, provided that the subject comprises one or more risk variants at the TNFSF15 locus, and provided that the anti-TL1A antibody comprises a heavy chain comprising complementarity determining regions (CDRs) of SEQ ID NOs: 6-8 and a light chain comprising complementarity determining regions (CDRs) of SEQ ID NOs: 14-16. In some embodiments, the anti-TL1A antibody is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the inflammatory bowel disease comprises Crohn's Disease, ulcerative colitis, medically refractive-ulcerative colitis, or a combination thereof.

In another aspect, provided herein is a method of treating an inflammatory bowel disease in a subject in need thereof, the method comprising administering to the subject an effective amount of an anti-TL1A antibody, provided that the subject comprises one or more risk variants at the TNFSF15 locus, and provided that the anti-TL1A antibody comprises a heavy chain comprising complementarity determining regions (CDRs) of SEQ ID NOs: 22-24 and a light chain comprising complementarity determining regions (CDRs) of SEQ ID NOs: 30-32. In some embodiments, the anti-TL1A antibody is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the inflammatory bowel disease comprises Crohn's Disease, ulcerative colitis, medically refractive-ulcerative colitis, or a combination thereof.

In another aspect, provided herein is a method of treating an inflammatory bowel disease in a subject in need thereof, the method comprising administering to the subject an effective amount of an anti-TL1A antibody, provided that the subject over-express TL1A, and provided that the anti-TL1A antibody comprises a heavy chain comprising complementarity determining regions (CDRs) of SEQ ID NOs: 6-8 and a light chain comprising complementarity determining regions (CDRs) of SEQ ID NOs: 14-16. In some embodiments, the anti-TL1A antibody is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the inflammatory bowel disease comprises Crohn's Disease, ulcerative colitis, medically refractive-ulcerative colitis, or a combination thereof.

In another aspect, provided herein is a method of treating an inflammatory bowel disease in a subject in need thereof, the method comprising administering to the subject an effective amount of an anti-TL1A antibody, provided that the subject over-express TL1A, and provided that the anti-TL1A antibody comprises a heavy chain comprising complementarity determining regions (CDRs) of SEQ ID NOs: 22-24 and a light chain comprising complementarity determining regions (CDRs) of SEQ ID NOs: 30-32. In some embodiments, the anti-TL1A antibody is: a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the inflammatory bowel disease comprises Crohn's Disease, ulcerative colitis, medically refractive-ulcerative colitis, or a combination thereof.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts agarose gel electrophoresis of total RNA from the hybridoma 684842-3 (5C3D11). DNA marker, Marker III, is shown in Lane M and Total RNA of 684842-3 is shown in Lane R.

FIG. 2 depicts agarose gel electrophoresis of PCR products of 684842-3. DNA marker, Marker III, is shown in Lane M, the variable heavy chain (VH) of 684842-3 is shown in Lane 1, and the variable light chain (VL) of 684842-3 is shown in Lane 2.

FIG. 3 depicts an agarose gel electrophoresis of total RNA from hybridoma 684842-6 (9E12E5). DNA marker, Marker III, is shown in Lame M and Total RNA of 684842-6 is shown in Lane R.

FIG. 4 depicts an agarose gel electrophoresis of PCR products of 684842-6. DNA marker, Marker III, is shown in Lane M, the VH of 684842-6 is shown in Lane 1, and the VL of 684842-6 is shown in Lane 2.

FIG. 5 demonstrates the inhibition of human TL1A induced IFN-γ production by 9E12E5 (panel A) and 5C3D11 (panel B).

FIG. 6 demonstrates the recognition capability of 5C3D11 (panel A) and 9E12E5 (panel B), to murine TL1A.

FIG. 7 depicts histogram graphs showing the fluorescent staining of 5C3D11 (panel A) and 9E12E5 (panel B) anti-TL1A antibodies on the TL1A expressing HEK293 cell line compared to the untransfected HEK293 cell line.

### DESCRIPTION OF THE INVENTION

Tumor necrosis factor-like protein 1A (TL1A) has been associated with the development and severity of severe colitis and Crohn's Disease. In addition, preclinical and human genetic association data suggests that TL1 A is a potential therapeutic target in Crohn's disease. The present disclosure describes neutralizing antibodies against TL1A and offers a novel therapeutic for the treatment of IBD.

All references cited herein are incorporated by reference in their entirety as though fully set forth. Unless defined otherwise, technical and scientific terms used herein have the same meaning as commonly understood by one of ordinary skill in the art. Singleton et al., Dictionary of Microbiology and Molecular Biology 3rd ed., Revised, J. Wiley & Sons (New York, NY 2006); and Sambrook and Russel, Molecular Cloning: A Laboratory Manual 4th ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2nd ed. (Cold Spring Harbor Press, Cold Spring Harbor NY, 2013); Kohler and Milstein, (1976) Eur. J. Immunol. 6:511; Queen et al. U.S. Pat. No. 5,585,089; and Riechmann et al., Nature 332:323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. Sep.; 23 (9): 1126-36).

One skilled in the art will recognize many methods and materials similar or equivalent to those described herein, which could be used to practice embodiments described herein. Indeed, this specification is not limited to the methods and materials described. Non-limiting definitions of select terms used herein are provided below.

“IBD” refers to Inflammatory Bowel Disease and includes, without limitation, Crohn's Disease, Ulcerative colitis and Medically Refractive Ulcerative colitis.

“CD”, “UC”, and “MR-UC” refer to Crohn's Disease, Ulcerative colitis, and Medically Refractive-Ulcerative colitis, respectively.

“TL1A” refers to TNF-like protein 1A.

“TNFSF15” refers to Tumor necrosis factor superfamily member 15, and is sometimes interchangeable with TL1A.

“SNP” refers to single nucleotide polymorphism.

“Risk variant” and risk allele” refer to an allele whose presence is associated with an increase in susceptibility to an inflammatory bowel disease, including but not limited to Crohn's Disease, Ulcerative colitis, and Medically Refractive-Ulcerative colitis, relative to an individual who does not have the risk variant or risk allele.

“Protective variant” and “protective allele” refer to an allele whose presence is associated with a decreased probability of developing inflammatory bowel disease, including but not limited to Crohn's Disease, Ulcerative colitis, and Medically Refractive-Ulcerative colitis, relative to an individual who does not have the protective variant or protective allele. The protective variant is more frequently present in healthy individuals compared to individuals diagnosed with inflammatory bowel disease.

“Protective” and “protection” as used with respect to the presence of particular specific variants or alleles refers to a decrease in susceptibility to IBD, including but not limited to CD, UC, and MR-UC.

“Risk” as used with respect to the presence of specific variants or alleles refers to an increase in susceptibility to IBD, including but not limited to CD, UC, and MR-UC.

“Biological sample” refers to any biological material from which nucleic acid and/or protein molecules can be found. As non-limiting examples, the term material encompasses whole blood, plasma, serum, saliva, cheek swab, and any other bodily fluid or tissue.

“IC” refers to immune complex.

“PBMC” refers to a peripheral blood mononuclear cell.

“Anti-TL1A therapy” refers to any reagent that suppresses a response to TL1A and/or inhibits TL1A signaling, including, without limitation, inhibition of any molecular signaling step from the TL1A ligand through its receptor to various upstream and/or downstream molecular targets. The anti-TL1A therapy can include the use of a small molecule; a nucleic acid such as siRNA, shRNA, and miRNA; a nucleic acid analogue such as PNA, pc-PNA, and LNA; an aptamer; a ribosome; a peptide; a protein; an avimer; an antibody, or variants and fragments thereof; and/or combinations of any thereof.

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgAQ1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” is inclusive of an “antigen-binding fragment” that refers to a portion of an antibody having antigenic determining variable regions of an antibody. Examples of antibody fragments include, but are not limited to Fab, Fab′, F(ab′)2, and Fv fragments, linear antibodies, single chain antibodies, and multispecific antibodies formed from antibody fragments.

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants.

The term “humanized antibody” refers to forms of non-human (e.g., murine) antibodies having specific immunoglobulin chains, chimeric immunoglobulins, or fragments thereof that contain minimal non-human (e.g., murine) sequences. For example, a humanized antibody comprises less than about 40% non-human sequence in the variable region. In some cases, a humanized antibody comprises less than about 20% non-human sequence in a full length antibody sequence. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). Examples of methods used to generate humanized antibodies are described in U.S. Pat. No. 5,225,539.

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising human light chain and human heavy chain polypeptides.

The term “chimeric antibodies” refers to antibodies wherein the sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

Each heavy and light chain is composed of a “variable region” of said heavy or light chain and a “constant region” of said heavy or light chain. The heavy chain and light chain regions may be further divided into hypervariable regions referred to as complementarity-determining regions (CDRs) and interspersed with conserved regions referred to as framework regions (FR). Each heavy chain and light chain region thus consists of three CDRs and four FRs, which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining region within antibody variable sequences and contributes to the formation of the antigen-binding site of antibodies. Techniques for determining CDRs are known in the art (e.g., Kabat et al. Sequences of Proteins of Immunological Interest, (5th ed., 1991, National Institutes of Health, Bethesda Md.; and Al-lazikani et al (1997) J. Molec. Biol. 273:927-948).

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate antigen binding are well-known in the art (see, e.g., Brummell et al., Biochem. 32:1180-1 187 (1993); Kobayashi et al. Protein Eng 12 (10): 879-884 (1999); and Burks et al. Proc. Natl. Acad. Sci. USA 94:412-417 (1997)).

That an antibody “specifically binds” to a protein means that the antibody reacts or associates more frequently, more rapidly, with greater duration, with greater affinity, or with some combination of the above to the protein than with alternative substances, including unrelated proteins. Because of the sequence identity between homologous proteins in different species, specific binding can include an antibody that recognizes a particular protein such as TL1A in more than one species. An antibody may in certain embodiments, bind to multiple targets bound by the same antigen-binding site on the antibody or the antibody may be bispecific and comprise at least two antigen-binding sites with differing specificities.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

The term “vector” means a construct, which is capable of delivering, and preferably expressing, one or more gene(s) and/or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure. In some cases, “substantially pure” refers to material which is at least 50% pure (i.e., free from contaminants), at least 90% pure, at least 95% pure, at least 98% pure, or at least 99% pure.

The terms “identical” or percent “identity” in the context of two or more nucleic acids or polypeptides, refer to two or more sequences or subsequences that are the same or have a specified percentage of nucleotides or amino acid residues that are the same, when compared and aligned for maximum correspondence. The percent identity may be measured using sequence comparison software or algorithms or by visual inspection. Various algorithms and software are known in the art that may be used to obtain alignments of amino acid or nucleotide sequences. Such algotithm/software programs include, but are not limited to NBLAST, XBLAST, Gapped BLAST, BLAST-2, WU-BLAST-2, ALIGN, ALIGN-2 or Megalign (DNASTAR).

Terms such as “treating” or “treatment” or “to treat” or “alleviating” or “to alleviate” refer to therapeutic treatment and/or prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. Thus, those in need of treatment include those already with the disorder; those prone to have the disorder; and those in whom the disorder is to be prevented.

The term “subject” refers to any animal (e.g., a mammal), including, but not limited to humans, non-human primates, rodents, and domestic and game animals, which is to be the recipient of a particular treatment. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Typically, the terms “subject” and “patient” are used interchangeably herein in reference to a human subject. In various embodiments, a subject can be one who has been previously diagnosed with or identified as suffering from or having a condition in need of treatment. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition.

The term “therapeutically effective amount” refers to an amount of an antibody, polypeptide, polynucleotide, small organic molecule, or other drug effective to “treat” a disease or disorder in a subject or mammal. In some cases, the therapeutically effective amount of the drug reduces the severity of IBD symptoms, including CD and UC/MR-UC symptoms. These include, but are not limited to, diarrhea, fever, fatigue, abdominal pain, abdominal cramping, inflammation, ulceration, nausea, vomiting, bleeding, blood in stool, reduced appetite, weight loss, and a combination thereof.

In one aspect, the disclosure describes the identification of two neutralizing anti-human TL1A monoclonal antibodies and five neutralizing humanized anti-human TL1A monoclonal antibodies. These antibodies neutralize the activity of TL1A in vitro and recognize both soluble and membrane bound TL1A.

TL1A (TNFSF15) is a TNF family member expressed mainly by endothelial cells, macrophages, and dendritic cells (DC). Its expression is induced by immune complex (IC) and cytokines. TL1A receptor DR3 is mainly expressed on T cells and NKT cells. In vitro, TL1A has been shown to enhance both human and mouse T cell proliferation and cytokine production. In vivo, TL1A transgenic mice developed IBD phenotype similar to human Crohn's. In addition, treatment of recombinant TL1 A protein also exacerbated colitis in mdr1−/−mice.

The present disclosure provides neutralizing anti-TL1A monoclonal antibodies useful for treating IBD, CD, UC and MR-UC. In some cases, these anti-TLA antibodies are used to treat a specific inflammatory bowel disease (IBD) patient population. Related polypeptides and polynucleotides, compositions comprising the anti-TL1A antibodies, and methods of making the anti-TL1A antibodies are also provided. Methods of using the novel anti-TL1A antibodies for treatment are further provided.

### Anti-TL1A Antibodies

Various embodiments provide antibodies that specifically bind to TL1A. In some embodiments, the antibodies specifically bind to soluble TL1A. In some embodiments, the antibodies specifically bind to membrane bound TL1A. The full-length amino acid (aa) sequence for the TL1A antibody “5C3D11” comprises: SEQ ID NO: 5 (heavy chain) and SEQ ID NO: 13 (light chain), as shown in Table 1. In various embodiments, the TL1A antibody comprises SEQ ID NO: 5 and SEQ ID NO: 13. A monomeric TL1A antibody comprises two instances of SEQ ID NO: 5 and SEQ ID NO: 13. In various embodiments, the TL1A antibody comprises SEQ ID NO: 6, SEQ ID NO: 7, SEQ ID NO: 8, SEQ ID NO: 14, SEQ ID NO: 15 and SEQ ID NO: 16, as shown in Table 1. In some embodiments, the TL1A antibody comprises at least one or any combination of SEQ ID NO: 6, SEQ ID NO: 7, SEQ ID NO: 8, SEQ ID NO: 14, SEQ ID NO: 15 and SEQ ID NO: 16.

The full-length nucleotide (nt) sequence for the TL1A antibody “5C3D11” is encoded by nucleic acid sequences comprising SEQ ID NO: 1 (heavy chain) and SEQ ID NO: 9 (light chain). In various embodiments, the TL1A antibody is encoded by nucleic acid sequences comprising SEQ ID NO: 1 and SEQ ID NO: 9, as shown in Table 1. A monomeric TL1A antibody is encoded by two instances of nucleic acid sequences comprising SEQ ID NO: 1 and SEQ ID NO: 9. In various embodiments, the TL1A antibody is encoded by nucleic acid sequences comprising SEQ ID NO: 2, SEQ ID NO: 3, SEQ ID NO: 4, SEQ ID NO: 10, SEQ ID NO: 11 and SEQ ID NO: 12, as shown in Table 1. In some embodiments, the TL1A antibody is encoded by at least one or any combination of nucleic acid sequences comprising SEQ ID NO: 2, SEQ ID NO: 3, SEQ ID NO: 4, SEQ ID NO: 10, SEQ ID NO: 11 and SEQ ID NO: 12.

The full-length amino acid (aa) sequence for the TL1A antibody “9E12E5” comprises: SEQ ID NO: 21 (heavy chain) and SEQ ID NO: 29 (light chain), as shown in Table 1. In various embodiments, the TL1A antibody comprises SEQ ID NO: 21 and SEQ ID NO: 29. A monomeric TL1A antibody comprises two instances of SEQ ID NO: 21 and SEQ ID NO: 29. In various embodiments, the TL1A antibody comprises SEQ ID NO: 22, SEQ ID NO: 23, SEQ ID NO: 24, SEQ ID NO: 30, SEQ ID NO: 31 and SEQ ID NO: 32, as shown in Table 1. In some embodiments, the TL1A antibody comprises at least one or any combination of SEQ ID NO: 22, SEQ ID NO: 23, SEQ ID NO: 24, SEQ ID NO: 30, SEQ ID NO: 31 and SEQ ID NO: 32.

The full-length nucleotide (nt) sequence for the TL1A antibody “9E12E5” is encoded by nucleic acid sequences comprising SEQ ID NO: 17 (TL1A heavy chain) and SEQ ID NO: 25 (TL1 A light chain), as shown in Table 1. In various embodiments, the TL1A antibody is encoded by nucleic acid sequences comprising SEQ ID NO: 17 and SEQ ID NO: 25. A monomeric TL1A antibody is encoded by two instances of nucleic acid sequences comprising SEQ ID NO: 17 and SEQ ID NO: 25. In various embodiments, the TL1A antibody is encoded by nucleic acid sequences comprising SEQ ID NO: 18, SEQ ID NO: 19, SEQ ID NO: 20, SEQ ID NO: 26, SEQ ID NO: 27 and SEQ ID NO: 28, as shown in Table 1. In some embodiments, the TL1A antibody is encoded by at least one or any combination of nucleic acid sequences comprising SEQ ID NO: 18, SEQ ID NO: 19, SEQ ID NO: 20, SEQ ID NO: 26, SEQ ID NO: 27 and SEQ ID NO: 28.

TABLE 1
Nucleotide and Amino Acid sequences for 5C3D11 and 9E12E5.

| | | | | | |
|---|---|---|---|---|---|
| | Sequence | | Leader | | |
| Antibody | Type | Size | Sequence | Sequence | SEQ ID No: |

5C3D11

| | | | | | |
|---|---|---|---|---|---|
| Heavy | DNA | 405 bp | FR1-CDR1- | ATGAAATGCAGCTGGGTTATCTTCTTCCTGATGGCAG | 1 |
| Chain | | | FR2-CDR2- | TGGTTACAGGGGTCAATTCAGAGGTTCAGCTGCAGCA | |
| | | | FR3-CDR3- | GTCTGGGGCAGAACTTGTGAAGCCAGGGGCCTCAGTC | |
| | | | FR4 | AAGTTGTCCTGCACAGCTTCTGGCTTCGACATTCAAG | |
| | | | | ACACCTATATGCACTGGGTGAAGCAGAGGCCTGAACA | |
| | | | | GGGCCTGGAGTGGATTGGAAGGATTGATCCTGCGAGT | |
| | | | | GGACATACTAAATATGACCCGAAGTTCCAGGTCAAGG | |
| | | | | CCACTATAACAACGGACACATCCTCCAACACAGCCTA | |
| | | | | CCTGCAGCTCAGCAGCCTGACATCTGAGGACACTGCC | |
| | | | | GTCTATTACTGTTCTAGATCGGGGGGCCTACCTGATG | |
| | | | | TCTGGGGCGCAGGGACCACGGTCACCGTCTCCTCA | |
| | | | CDR1 | GACACCTATATGCAC | 2 |
| | | | CDR2 | AGGATTGATCCTGCGAGTGGACATACTA | 3 |
| | | | | AATATGACCCGAAGTTCCAGGTC | |
| | | | CDR3 | TCGGGGGGCCTACCTGATGTC | 4 |
| Heavy | Amino | 135 aa | FR1-CDR1- | MKCSWVIFFLMAVVTGVNSEVQLQQSGAELVKPGASV | 5 |
| Chain | Acid | | FR2-CDR2- | KLSCTASGFDIQDTYMHWVKQRPEQGLEWIGRIDPAS | |
| | | | FR3-CDR3- | GHTKYDPKFQVKATITTDTSSNTAYLQLSSLTSEDTA | |
| | | | FR4 | VYYCSRSGGLPDVWGAGTTVTVSS | |
| | | | CDR1 | DTYMH | 6 |
| | | | CDR2 | RIDPASGHTKYDPKFQV | 7 |
| | | | CDR3 | SGGLPDV | 8 |
| Light | DNA | 384 bp | FR1-CDR1- | ATGGATTTTCAAGTGCAGATTTTCAGCTTCCTGCTAA | 9 |
| Chain | | | FR2-CDR2- | TCAGTGCTTCAGTCATAATGTCCAGAGGACAAATTGT | |
| | | | FR3-CDR3- | TCTCTCCCAGTCTCCTGCAATCCTGTCTGCATCTCCA | |
| | | | FR4 | GGGGAGAAGGTCACAATGACTTGCAGGGCCAGCTCAA | |
| | | | | GTGTAAGTTACATGTACTGGTACCAGCAGAAGCCTGG | |
| | | | | ATCCTCCCCCAAACCCTGGATTTATGCCACATCCAAC | |
| | | | | CTGGCTTCTGGAGTCCCTGATCGCTTCAGTGGCAGTG | |
| | | | | GGTCTGGGACCTCTTACTCTCTCACAATCAGCAGAGT | |
| | | | | GGAGGCTGAAGATGCTGCCACTTATTACTGCCAGCAG | |
| | | | | TGGAGTGGTAACCCACGGACGTTCGGTGGAGGCACCA | |
| | | | | AGCTGGAAATCAAA | |
| | | | CDR1 | AGGGCCAGCTCAAGTGTAAGTTACATGTAC | 10 |
| | | | CDR2 | GCCACATCCAACCTGGCTTCT | 11 |
| | | | CDR3 | CAGCAGTGGAGTGGTAACCCACGGACG | 12 |
| Light | Amino | 128 aa | FR1-CDR1- | MDFQVQIFSFLLISASVIMSRGQIVLSQSPAILSASP | 13 |
| Chain | Acid | | FR2-CDR2- | GEKVIMTCRASSSVSYMYWYQQKPGSSPKPWIYATSN | |
| | | | FR3-CDR3- | LASGVPDRFSGSGSGTSYSLTISRVEAEDAATYYCQQ | |
| | | | FR4 | WSGNPRTFGGGTKLEIK | |
| | | | CDR 1 | RASSSVSYMY | 14 |
| | | | CDR2 | ATSNLAS | 15 |
| | | | CDR3 | QQWSGNPRT | 16 |

9E12E5

| | | | | | |
|---|---|---|---|---|---|
| Heavy | DNA | 405 bp | FR1-CDR1- | ATGGGATGGAGCTGGGTCTTTCTCTTCCTCCTGTCAG | 17 |
| Chain | | | FR2-CDR2- | TGACTGCAGGTGTCCACTCCCAGGTTCACCTGCAGCA | |
| | | | FR3-CDR3- | GTCTGGACCTGAACTGGTAAAGCCTGGGGCTTCAGTG | |
| | | | FR4 | AAGTTGTCCTGCAAGGCTTCTGGCTACACCTTCACAA | |
| | | | | AGTATGATATAAACTGGGTGAGGCAGAGGCCTGAACA | |
| | | | | GGGACTTGAGTGGATTGGATGGATTTTTCCTGGAGAT | |
| | | | | GGTAGAACTGACTACAATGAGAAGTTCAAGGGTAAGG | |
| | | | | CCACACTGACTACAGACAAATCCTCCAGCACAGCCTA | |
| | | | | CATGGAGGTCAGCAGGCTGACATCTGAGGACTCTGCT | |
| | | | | GTCTATTTCTGTGCAAGATATGGCCCCGCTATGGACT | |
| | | | | ACTGGGGTCAAGGAACCTCAGTCACCGTCGCCTCA | |
| | | | CDR1 | AAGTATGATATAAAC | 18 |
| | | | CDR2 | TGGATTTTTCCTGGAGATGGTAGAACTGACTACAATG | 19 |
| | | | | AGAAGTTCAAGGGT | |
| | | | CDR3 | TATGGCCCCGCTATGGACTA | 20 |
| Heavy | Amino | 135 aa | FR1-CDR1- | MGWSWVFLFLLSVTAGVHSQVHLQQSGPELVKPGASV | 21 |
| Chain | Acid | | FR2-CDR2- | KLSCKASGYTFTKYDINWVRQRPEQGLEWIGWIFPGD | |
| | | | FR3-CDR3- | GRTDYNEKFKGKATLTTDKSSSTAYMEVSRLTSEDSA | |
| | | | FR4 | VYFCARYGPAMDYWGQGTSVTVAS | |
| | | | CDR1 | KYDIN | 22 |
| | | | CDR2 | WIFPGDGRTDYNEKFKG | 23 |
| | | | CDR3 | YGPAMDY | 24 |
| Light | DNA | 393 bp | FR1-CDR1- | ATGAAGTTGCCTGTTAGGCTGTTGGTGCTGATGTTCT | 25 |
| Chain | | | FR2-CDR2- | GGATTCCTGCTTCCAGCAGTGATGTTTTGATGACCCA | |
| | | | FR3-CDR3- | AACTCCACTCTCCCTGCCTGTCAGTCTTGGAGATCAA | |
| | | | FR4 | GCCTCCATCTCTTGCAGATCTAGTCAGACCATTGTAC | |
| | | | | ATAGTAATGGAGACACCTATTTAGACTGGTTCCTGCA | |
| | | | | GAAACCAGGCCAGTCTCCAAAGCTCCTGATCTACAAA | |
| | | | | GTTTCCAACCGATTTTCTGGGGTCCCAGACAGGTTCA | |
| | | | | GTGGCAGTGGATCAGGGACAGATTTCACACTCAAGAT | |
| | | | | CAGCAGAGTGGAGGCTGAGGATCTGGGAGTTTATTAC | |
| | | | | TGCTTTCAAGGTTCACATGTTCCGTACACGTTCGGAG | |
| | | | | GGGGGACCAAGCTGGAAATAAAA | |
| | | | CDR1 | AGATCTAGTCAGACCATTGTACATAGTAATGGAGACA | 26 |
| | | | | CCTATTTAGAC | |
| | | | CDR2 | AAAGTTTCCAACCGATTTTCT | 27 |
| | | | CDR3 | TTTCAAGGTTCACATGTTCCGTACACG | 28 |
| Light | Amino | 13l aa | FR1-CDR1- | MKLPVRLLVLMFWIPASSSDVLMTQTPLS | 29 |
| Chain | Acid | | FR2-CDR2- | LPVSLGDQASISCRSSQTIVHSNGDTYLD | |
| | | | FR3-CDR3- | WFLQKPGQSPKLLIYKVSNRFSGVPDRFSG | |
| | | | FR4 | SGSGTDFTLKISRVEAEDLGVYYCFQGSHV | |
| | | | | PYTFGGGTKLEIK | |
| | | | CDR1 | RSSQTIVHSNGDTYLD | 30 |
| | | | CDR2 | KVSNRFS | 31 |
| | | | CDR3 | FQGSHVPYT | 32 |

In various embodiments, the anti-TL1A antibody binds specifically to the same region of TL1A or binds specifically to a region of TL1A that overlaps with the region of TL1A to which an antibody comprising a heavy chain variable region encoded by SEQ ID NO:1 and a light chain variable region encoded by SEQ ID NO: 9 binds specifically. In various other embodiments, the anti-TL1A antibody binds specifically to the same region of TL1A or binds specifically to a region of TL1A that overlaps with the region of TL1A to which an antibody comprising a heavy chain comprising the sequence of SEQ ID NO: 5 and a light chain comprising the sequence of SEQ ID NO: 13 binds specifically. In other embodiments, the anti-TL1A antibody binds specifically to the same region of TL1A or binds specifically to a region of TL1A that overlaps with the region of TL1A to which an antibody comprising a heavy chain variable region encoded by SEQ ID NO: 17 and a light chain variable region encoded by SEQ ID NO: 25 binds specifically. In certain other embodiments, the anti-TL1A antibody binds specifically to the same region of TL1A or binds specifically to a region of TL1A that overlaps with the region of TL1A to which an antibody comprising a heavy chain comprising SEQ ID NO: 21 and a light chain comprising SEQ ID NO: 29 binds specifically.

Further embodiments provide polypeptides, including, but not limited to, antibodies that specifically bind to TL1A, that comprise one, two, three, four, five, six, seven, eight, nine, ten, eleven and/or twelve of the CDRs of 5C3D11 and/or 9E12E5 (see Table 1, and Tables 2 and 3 of Example 1 and 2 below). In certain embodiments, the polypeptides comprise the heavy chain CDRs of 5C3D11 (SEQ ID NOs: 6, 7, and 8) and/or 9E12E5 (SEQ ID NOs: 22, 23, and 24), the light chain CDRs of 5C3D11 (SEQ ID NOs: 14, 15, and 16) and/or 9E12E5 (SEQ ID NOs: 30, 31, and 32), or combinations thereof. In certain other embodiments, the heavy chain CDR(s) are contained within a heavy chain variable region and/or the light chain CDR(s) are contained within a light chain variable region. In some embodiments, polypeptides comprising one of the individual light chains or heavy chains described herein, as well as polypeptides (e.g., antibodies) comprising both a light chain and a heavy chain are also provided. In some embodiments, the anti-TL1A antibody comprises the heavy chains and light chains of 5C3D11. In other embodiments, the anti-TL1A antibody comprises the heavy chains and light chains of 9E12E5. In certain embodiments, each CDR in the anti-TL1A antibody comprises up to four (i.e., 0, 1, 2, 3, or 4) conservative amino acid substitutions per CDR.

In various embodiments, the anti-TL1A antibody or fragment has a binding affinity to TL1A of at least about 1E⁻⁷, 1E⁻⁸, 1E⁻⁹, 1E⁻¹⁰, or 1E⁻¹¹. In some cases, the binding affinity is from about 1E⁻⁹ to about 1E⁻¹¹. For example, in some cases the binding affinity is about 7.90E⁻¹¹. In various embodiments, the anti-TL1A antibody or fragment has a binding affinity to TL1A of about 7.90E⁻⁹ to about 7.90E⁻¹⁰. In various embodiments, the anti-TL1A antibody or fragment has a binding affinity to TL1A of about 7.90E⁻¹⁰ to about 7.90E⁻¹². In various embodiments, the anti-TL1A antibody or fragment has a binding affinity to TL1A of about 7.90E⁻¹² to about 7.90E⁻¹³.

In various embodiments, the anti-TL1A antibody or fragment has a binding affinity to TL1A of about 5.20E⁻¹¹. In various embodiments, the anti-TLA antibody or fragment has a binding affinity to TL1A of about 5.20E⁻⁹ to about 5.20E⁻¹⁰. In various embodiments, the anti-TL1A antibody or fragment has a binding affinity to TL1A of about 5.20E⁻¹⁰ to 5.20E⁻¹². In various embodiments, the anti-TL1A antibody or fragment has a binding affinity to TL1A of about 5.20E⁻¹² to 5.20E^{-13.}

Various embodiments provide for an anti-TL1A antibody that binds to the same region of a TL1A protein or portion thereof as a reference antibody, e.g., any anti-TL1A antibody described herein. In some embodiments, the reference antibody comprises the heavy chain CDRs of SEQ ID NOs: 6-8 and the light chain CDRs of SEQ ID NOs: 14-16. In some cases, the reference antibody comprises a heavy chain variable domain of SEQ ID NO: 5 and a light chain variable domain of SEQ ID NO: 13. In some embodiments, the reference antibody comprises the heavy chain CDRs of SEQ ID NOs: 22-24 and the light chain CDRs of SEQ ID NOs: 30-32. In some cases, the reference antibody comprises a heavy chain variable domain of SEQ ID NO: 21 and a light chain variable domain of SEQ ID NO: 29. In some cases, the reference antibody is 5C3D11. In some cases, the reference antibody is 9E12E5.

Non-limiting methods for determining whether an anti-TL1A antibody (i.e. test antibody) binds to the same region of a TL1A protein or portion thereof as an antibody described herein are provided. An exemplary embodiment comprises a competition assay. For instance, the method comprises determining whether the test antibody can compete with binding between the reference antibody and the TL1A protein or portion thereof, or determining whether the reference antibody can compete with binding between the test antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay. Non-limiting methods are described in Example 9 and Example 10.

### Methods of Generating Antibodies

Various embodiments provide for an antibody that is generated using a polypeptide or a nucleotide sequence. In some embodiments, the antibody is a monoclonal antibody. In some embodiments, the antibody is a human antibody or a humanized antibody. In some embodiments, the antibody is an antibody fragment. For example, the antibody is a Fab. In some embodiments, the antibody is a chimeric antibody.

The antibodies described herein can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York).

In various embodiments, the antibody is an antagonist of a TL1A receptor, such as, but not limited to, DR3 and TR6/DcR3. In certain embodiments, the antibody inhibits at least about 10%, at least about 20%, at least about 30%, at least about 50%, at least about 75%, at least about 90%, or about 100% of one or more activity of the bound TL1A receptor.

In various embodiments, monoclonal antibodies are prepared using methods known in the art, such as, but not limited to the hybridoma method, where a host animal is immunized, as described above to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, monoclonal antibodies are made using recombinant DNA methods as described in U.S. Pat. No. 4,816,567. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, “chimeric antibodies”, a molecule in which different portions are derived from different animal species, such as those having a variable region derived from a murine monoclonal antibody and a human immunoglobulin constant region (e.g., humanized antibodies) can be generated. Chimeric antibodies can be produced using various techniques known in the art (see Morrison et al., Proc. Natl. Acad. Sci. 81:851-855 (1984); Neuberger et al., Nature 312:604-608 (1984); Takeda et al., Nature 314:452-454 (1985)).

In some embodiments, the anti-TL1A monoclonal antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody (see, e.g., U.S. Pat. Nos. 5,585,089; 6,835,823; 6,824,989). In various embodiments, a humanized antibody can be further optimized to decrease potential immunogenicity, while maintaining functional activity, for therapy in humans.

In some embodiments, the humanized anti-TL1A antibody comprises a heavy chain variable domain of any of SEQ ID NOS: 35-39. In some embodiments, the humanized anti-TL1A antibody comprises a light chain variable domain of any of SEQ ID NOS: 40-44. In some cases, the humanized anti-TL1A antibody comprises a heavy chain variable domain having SEQ ID NO: 35, and a light chain variable domain having SEQ ID NO: 40. In some cases, the humanized anti-TL1A antibody comprises a heavy chain variable domain having SEQ ID NO: 36, and a light chain variable domain having SEQ ID NO: 41. In some cases, the humanized anti-TL1A antibody comprises a heavy chain variable domain having SEQ ID NO: 37, and a light chain variable domain having SEQ ID NO: 42. In some cases, the humanized anti-TL1A antibody comprises a heavy chain variable domain having SEQ ID NO: 38, and a light chain variable domain having SEQ ID NO: 43. In some cases, the humanized anti-TL1A antibody comprises a heavy chain variable domain having SEQ ID NO: 39, and a light chain variable domain having SEQ ID NO: 44.

In certain embodiments, the anti-TL1A antibody is a human antibody. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boerner et al., 1991, J. Immunol., 147 (1): 86-95; and U.S. Pat. No. 5,750,373). A human antibody can be selected from a phage library. Techniques for the generation and use of antibody phage libraries are described in U.S. Pat. Nos. 5,969,108, 6, 172, 197, 5,885, 793, 6, 521,404; 6,544, 731; 6,555,313; 6,582,915; 6,593,081; 6,300,064; 6,653,068; 6,706,484; and 7,264,963; and Rothe et al., 2007, J. Mol. Bio., doi: 10.1016/j.jmb.2007.12.018.

Humanized antibodies can also be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625, 126; 5,633,425; and 5,661,016. A humanized antibody may also be obtained by a novel genetic engineering approach that enables production of affinity-matured human-like polyclonal antibodies in large animals such as, for example, rabbits and mice. (See, e.g. U.S. Pat. No. 6,632,976).

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g. mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR. Methods to obtain “humanized antibodies” are well known to those skilled in the art. (e.g., U.S. Pat. Nos. 5,861,155, 6,479,284, 6,407,213, 5,624,821, US2003166871, US20020078757, Queen et al., Proc. Natl. Acad Sci USA, 86:10029-10032 (1989) and Hodgson et al., Bio/Technology, 9:421 (1991)).

Chimeric, humanized and human antibodies are typically produced by recombinant expression. Recombinant polynucleotide constructs typically include an expression control sequence operably linked to the coding sequences of antibody chains, including naturally-associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate IBD. Various techniques are known for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A (see e.g., U.S. Pat. No. 4,946,778). In addition, methods can be adapted for the construction of Fab expression libraries (see e.g., Huse, et al., Science 246:1275-1281 (1989)) to allow rapid and effective identification of monoclonal Fab fragments with the desired specificity for TL1A, or derivatives, fragments, analogs or homologs thereof. Antibody fragments may be produced by techniques in the art including, but not limited to: (a) a F(ab′)2 fragment produced by pepsin digestion of an antibody molecule; (b) a Fab fragment generated by reducing the disulfide bridges of an F(ab′) 2 fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, NY, 1979 and 1981).

Various embodiments provide for a genetic construct comprising a nucleic acid encoding an anti-TL1A antibody or fragment provided herein. Genetic constructs of the antibody can be in the form of expression cassettes, which can be suitable for expression of the encoded anti-TL1A antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

### Polypeptides and Polymicleotides

Various embodiments provide for a polypeptide comprising one or more complementarity determining regions selected from the group consisting of SEQ ID NO: 6, SEQ ID NO: 7, SEQ ID NO: 8, SEQ ID NO: 14, SEQ ID NO: 15, SEQ ID NO: 16, SEQ ID NO: 22, SEQ ID NO: 23, SEQ ID NO: 24, SEQ ID NO: 30, SEQ ID NO: 31; and SEQ ID NO: 32. In various embodiments, the polypeptide comprises 2, 3, 4, 5, 6, 7, 8, 9, 10, 11 or 12 of these complementarity determining regions.

In various embodiments, the antibody is generated using a polypeptide or a polynucleotide.

The polypeptides can be recombinant polypeptides, or synthetic polypeptides comprising an antibody, or fragment thereof, against TL1A. When specifically noted, polypeptides can be natural polypeptides. It will be recognized in the art that some amino acid sequences can be varied without significant effect of the structure or function of the protein. Thus, further provided are variations of the polypeptides which show substantial activity or which include regions of an antibody, or fragment thereof, against a TL1A protein. Such modifications include deletions, insertions, inversions, repeats, and type substitutions.

The isolated polypeptides described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine (SEQ ID NO: 45), maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated. Methods known in the art for purifying antibodies and other proteins also include, for example, those described in U.S. Patent Publication No. 2008/0177048, and 2009/0187005.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A “variant,” as referred to herein with respect to a polypeptide, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques known to one of skill in the art. Mutations can be introduced at particular loci or by oligonucleotide-directed site-specific mutagenesis procedures. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462.

In certain embodiments, provided are polynucleotides that encode a polypeptide that specifically binds TL1A or a fragment thereof. For example, provided is a polynucleotide comprising a nucleic acid sequence that encodes an antibody to TL1A or encodes a fragment of such an antibody. The polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand. In certain embodiments, the polynucleotides are isolated. In certain embodiments, the polynucleotides are substantially pure.

Further provided is a polynucleotide comprising a sequence selected from the group consisting of SEQ ID NOs: 1, 9, 17, 25 and a combination thereof. Also provided are variants of the herein described polynucleotides encoding, for example, fragments, analogs, and derivatives.

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector,” it is meant that the vector includes a heterologous nucleic acid sequence, or “transgene” that is capable of expression in vivo.

In certain embodiments, provided are isolated polynucleotides comprising polynucleotides having a nucleotide sequence at least 80% identical, at least 85% identical, at least 90% identical, at least 95% identical, and in some embodiments, at least 96%, 97%, 98% or 99% identical to a polynucleotide encoding a polypeptide comprising an antibody, or fragment thereof, to TL1A described herein.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

In some embodiments, genes are fused to create a desired sequence. Each fused gene is assembled in or inserted into an expression vector, which is transfected into a recipient. The transfected recipient cells are cultured under conditions that permit expression of the incorporated genes and the expressed immunoglobulin chains or intact antibodies or fragments are recovered from the culture. In some embodiments, the fused genes encoding the antibody, antigen-binding fragment thereof, are assembled in separate expression vectors that are then used to co-transfect a recipient cell.

### Pharmaceutical Compositions, Administration and Dosage

The anti-TL1A antibodies provided are useful in a variety of applications including, but not limited to, therapeutic treatment methods, such as the treatment of IBD. The methods of use may be in vitro, ex vivo, or in vivo methods. In certain embodiments, the anti-TL1A antibody is an antagonist for TL1A receptors.

In certain embodiments, the disease treated with anti-TL1A antibody or TL1A receptor antagonist is IBD, CD, UC and/or MR-UC.

In various embodiments, the pharmaceutical compositions are formulated for delivery via any route of administration. “Route of administration” may refer to any administration pathway known in the art, including but not limited to aerosol, nasal, oral, transmucosal, transdermal or parenteral.

“Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

Via the topical route, the pharmaceutical compositions are formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

Via the ocular route, they may be in the form of eye drops.

In various embodiments, an agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. In particular embodiments, compounds used herein are administered orally, intravenously or intramuscularly to a patient having IBD, CD, UC and/or MR-UC.

The pharmaceutical compositions can also contain any pharmaceutically acceptable carrier. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of an anti-TL1A antibody. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in the therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain minor amounts of auxiliary substances such as wetting or emulsifying agents, pH buffering agents and the like which enhance or maintain the effectiveness of the active ingredient. The therapeutic composition as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of an active agent (i.e. antibody or fragment thereof) used that will be effective in the treatment of a particular disorder or condition will depend on the nature of the disorder or condition, and can be determined by one of skill in the art with standard clinical techniques.

The pharmaceutical compositions can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective anti-TL1A antibody can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biological samples obtained, or the responses observed in the appropriate animal models.

For the treatment of the disease, the appropriate dosage of an antibody depends on the type of disease to be treated, the severity and course of the disease, the responsiveness of the disease, whether the antibody is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates. The TL1A antibody can be administered one time or over a series of treatments lasting from several days to several months, or until a cure is effected or a diminution of the disease state is achieved (e.g., treatment or amelioration of IBD). The duration of treatment depends upon the subject's clinical progress and responsiveness to therapy. In certain embodiments, dosage is from 0.01 μg to 100 mg per kg of body weight, and can be given once or more daily, weekly, monthly or yearly. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

### Methods of Treatment

Various embodiments provide for methods of treating inflammatory bowel disease (IBD), comprising administering an anti-TL1A antibody described herein to a subject in need thereof. In some embodiments, the subject comprises one or more risk variants.

In various embodiments, provided herein is a method of treating inflammatory bowel disease (IBD) in a subject in need thereof, comprising: administering to the subject a therapeutically effective amount of an antibody or an antigen-binding fragment that specifically binds TL1A, wherein the antibody or antigen-binding fragment is selected from the group consisting of: (a) a heavy chain comprising SEQ ID NO: 5 and a light chain comprising SEQ ID NO: 13; (b) a heavy chain comprising SEQ ID NO: 21 and a light chain comprising SEQ ID NO: 29; (c) the heavy chain complementarity determining regions (CDRs) of SEQ ID NOs: 6-8 and the light chain complementarity determining regions (CDRs) of SEQ ID NOs: 14-16; (d) the heavy chain complementarity determining regions (CDRs) of SEQ ID NOs: 22-24 and the light chain complementarity determining regions (CDRs) of SEQ ID NOs: 30-32; (e) the heavy chain of SEQ ID NOs: 5 and/or 21; (f) the light chain of SEQ ID NOs: 13 and/or 29; (g) the heavy chain complementarity determining regions (CDRs) of SEQ ID NOs: 6-8 and/or SEQ ID NOs: 22-24; (h) the light chain complementarity determining regions (CDRs) of SEQ ID NOs: 14-16 and/or SEQ ID NOs: 30-32; and (i) combinations thereof.

In various embodiments, inflammatory bowel disease (IBD) is Crohn's Disease, ulcerative colitis, and/or medically refractive-ulcerative colitis.

In various embodiments, the antibody is a monoclonal antibody. In some embodiments, the antibody is a human antibody. In various embodiments, the antibody is a humanized antibody. In various embodiments, the antibody is a neutralizing antibody.

In various other embodiments, the subject is determined to have an increased TL1A expression. In some embodiments, the administration of a therapeutically effective amount of an anti-TL1A antibody causes a decrease in TL1A in the subject treated.

In various embodiments, the antibody or antigen-binding fragment is selected from the group consisting of: (a) a heavy chain comprising SEQ ID NO: 5 and a light chain comprising SEQ ID NO: 13; (b) the heavy chain complementarity determining regions (CDRs) of SEQ ID NOs: 6-8 and the light chain complementarity determining regions (CDRs) of SEQ ID NOs: 14-16; (c) the heavy chain of SEQ ID NO: 5; (d) the light chain of SEQ ID NO: 13; (e) the heavy chain complementarity determining regions (CDRs) of SEQ ID NOs: 6-8; (f) the light chain complementarity determining regions (CDRs) of SEQ ID NOs: 14-16; and (g) combinations thereof.

In various other embodiments, the antibody or antigen-binding fragment is selected from the group consisting of: (a) a heavy chain comprising SEQ ID NO: 21 and a light chain comprising SEQ ID NO: 29; (b) the heavy chain complementarity determining regions (CDRs) of SEQ ID NOs: 22-24 and the light chain complementarity determining regions (CDRs) of SEQ ID NOs: 30-32; (c) the heavy chain of SEQ ID NO: 21; (d) the light chain of SEQ ID NO: 29; (e) the heavy chain complementarity determining regions (CDRs) of SEQ ID NOs: 22-24; (f) the light chain complementarity determining regions (CDRs) of SEQ ID NOs: 30-32; and (g) combinations thereof.

In various embodiments, the antibody or antigen-binding fragment is encoded by a nucleic acid having a sequence selected from the group consisting of SEQ ID NO: 1, SEQ ID NO: 9, SEQ ID NO: 17, SEQ ID NO: 25 and combinations thereof.

In various other embodiments, the antibody or antigen-binding fragment is encoded by a nucleic acid having a sequence selected from the group consisting of: (a) the heavy chain complementarity determining regions (CDRs) of SEQ ID NOs: 2-4 and the light chain complementarity determining regions (CDRs) of SEQ ID NOs: 10-12; (b) the heavy chain complementarity determining regions (CDRs) of SEQ ID NOs: 18-20 and the light chain complementarity determining regions (CDRs) of SEQ ID NOs: 26-28; (c) the heavy chain of SEQ ID NOs: 1 and/or 17; (d) the light chain of SEQ ID NOs: 9 and/or 25; (e) the heavy chain complementarity determining regions (CDRs) of SEQ ID NOs: 2-4 and/or SEQ ID NOs: 18-20; (f) the light chain complementarity determining regions (CDRs) of SEQ ID NOs: 10-12 and/or SEQ ID NOs: 26-28; and (g) combinations thereof.

In various aspects, the anti-TL1A antibody is administered to the subject for treatment. In various other embodiments, the anti-TL1A antibody is administered in a series of treatments. In some embodiments, the anti-TL1A antibody and a second IBD treatment may be administered in any order or concurrently. In selected embodiments, the anti-TL1A antibody will be administered to patients that have previously undergone treatment with the second IBD treatment. In certain other embodiments, the anti-TL1A antibody and the second IBD treatment will be administered substantially simultaneously or concurrently. For example, a subject may be given the anti-TL1A antibody while undergoing a course of treatment with the second IBD treatment. In certain embodiments, the anti-TL1A antibody will be administered within 1 year of the treatment with the second IBD treatment. In certain alternative embodiments, the anti-TL1A antibody will be administered within 10, 8, 6, 4, or 2 months of any treatment with the second IBD treatment. In certain other embodiments, the anti-TL1A antibody will be administered within 4, 3, 2, or 1 week of any treatment with the second IBD treatment. In some embodiments, the anti-TL1A antibody will be administered within 5, 4, 3, 2, or 1 days of any treatment with the second IBD treatment. It will further be appreciated that the two treatments may be administered to the subject within a matter of hours or minutes (i.e., simultaneously).

Other IBD treatments include, but are not limited to 1) anti-inflammatory drugs (e.g., Aminosalicylates such as, but not limited to sulfasalazine Azulfidine, 5-aminosalicylates, Mesalamine, Asacol, Lialda, Rowasa, Canasa, balsalazide Colazal and olsalazine, Dipentum); 2) corticosteroids (e.g., prednisone and hydrocortisone); 3) immune system suppressors (e.g., Azathioprine, Azasan, Imuran, mercaptopurine, Purinethol, Purixan, Cyclosporine, Gengraf, Neoral and Sandimmune, Infliximab, Remicade, adalimumab, Humira, golimumab, and Simponi, tumor necrosis factor (TNF)-alpha inhibitors (e.g., Infliximab), Methotrexate, Rheumatrex, Natalizumab, Tysabri, vedolizumab, Entyvio, Ustekinumab and Stelara; 4) Antibiotics (e.g., Metronidazole, Flagyl, Ciprofloxacin, Cipro); 5) Anti-diarrheal medications (e.g., fiber supplements-Metamucil or Citrucel) or loperamide; 6) Pain relievers (e.g., Tylenol, ibuprofen, naproxen sodium and diclofenac sodium); and 7) Surgery (e.g., removal of the colon, partial digestive tract removal, colectomy, proctocolectomy and/or strictureplasty). In some embodiments, these IBD treatments may be administered in combination with the anti-TL1A antibody. Treatment with an antibody can occur prior to, concurrently with, or subsequent to administration of an IBD treatment. Combined administration can include co-administration, either in a single pharmaceutical formulation or using separate formulations, or consecutive administration in either order but generally within a time period such that all active agents can exert their biological activities simultaneously. Any dosing schedules for such IBD treatments can also be used as determined by the skilled practitioner.

In some embodiments, the second IBD treatment comprises an antibody. Thus, treatment can involve the combined administration of antibodies provided herein with other antibodies against additional IBD-associated antigens, such as, but not limited to tumor necrosis factor (TNF)-alpha. Combined administration can include co-administration, either in a single pharmaceutical formulation or using separate formulations, or consecutive administration in either order but generally within a time period such that all active agents can exert their biological activities simultaneously.

### Kits

Further provided is a kit to treat IBD (e.g., CD, UC and/or MR-UC). The kit comprises of the antibodies described herein, which can be used to perform the methods described herein. The kit is useful for practicing the inventive method of providing treatment to an IBD, CD, UC and/or MR-UC patient by administering an anti-TL1A antibody. The kit is an assemblage of materials or components, including at least one of the inventive compositions. Thus, in some embodiments the kit contains a composition including anti-TL1A antibodies, for the treatment of IBD, CD, UC and/or MR-UC, as described above. In other embodiments, the kits contains all of the components necessary and/or sufficient to perform a detection assay for TL1A, including all controls, directions for performing assays, and any necessary software for analysis and presentation of results.

The exact nature of the components configured in the inventive kit depends on its intended purpose. For example, some embodiments are configured for the purpose of treating IBD, CD, UC and/or MR-UC. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or alleviate IBD, CD, UC and/or MR-UC. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of an inventive composition containing anti-TL1 A antibodies and/or primers and probes for TL1A. The packaging material generally has an external label which indicates the contents and/or purpose of the kit and/or its components.

### EXAMPLES

The following examples are illustrative of the embodiments described herein and are not to be interpreted as limiting the scope of the described embodiments. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1

### Generation of Immunogen and Immunization Protocol for Hybridoma Generation

A recombinant TL1A protein having a point mutation C66S (as counted from the leading methionine in the full length protein) and lacking the leading 57 amino acids was expressed in E. coli. The recombinant TL1A sequence is represented by SEQ ID NO: 33

(QLRAQGEASVQFQALKGQEFAPSHQQVYAPLRADGDKPRAHLTVVRQTP
TQHFKNQFPALHWEHELGLAFTKNRMNYTNKFLLIPESGDYFIYSQVTFR
GMTSECSEIRQAGRPNKPDSITVVITKVTDSYPEPTQLLMGTKSVCEVGS
NWFQPIYLGAMFSLQEGDKLMVNVSDISLVDYTKEDKTFFGAFLL).

A TL1A expressing HEK293 cell line was generated by transduction with a lenti viral construct containing the sequence for the full length TL1A protein. This cell line expresses both the membrane bound and secreted forms of the protein (as confirmed by flow cytometry and ELISA based methods respectively). The sequence of TL1A expressed by the HEK293 Cell Line is represented by SEQ ID NO: 34

(MAEDLGLSFGETASVEMLPEHGSCRPKARSSSARWALTCCLVLLPFLAG
LTTYLLVSQLRAQGEACVQFQALKGQEFAPSHQQVYAPLRADGDKPRAHL
TVVRQTPTQHFKNQFPALHWEHELGLAFTKNRMNYTNKFLLIPESGDYFI
YSQVTFRGMTSECSEIRQAGRPNKPDSITVVITKVTDSYPEPTQLLMGTK
SVCEVGSNWFQPIYLGAMFSLQEGDKLMVNVSDISLVDYTKEDKTFFGAF
LL).

Mice were immunized with multiple rounds of immunization using both the recombinant protein and the TL1A expressing cell line. The strategy involved a primary immunization and two rounds of boosters. Two groups of animals were immunized, swapping the primary and booster immunogens between the groups: group 1: primary immunization with recombinant TL1A protein and boost with TL1A expressing cell line; and group 2: primary immunization with TL1A expressing cell line and boost with recombinant TL1A protein. B cells removed from the mice were fused with myeloma cells to generate hybridomas expressing 5C3D11 and 9E12E5 antibodies.

### Monoclonal Antibody Sequencing of Hybridoma 5C3D11

Total RNA was extracted from frozen hybridoma cells and cDNA was synthesized from the RNA. PCR was then performed to amplify the variable regions (heavy and light chains) of the antibody, which were then cloned into a standard cloning vector separately and sequenced.

### Materials and Methods

Materials used include hybridoma cells, TRIzol® Reagent (Ambion, Cat. No.: 15596-026), and PrimeScript™ 1 st Strand cDNA Synthesis Kit (Takara, Cat. No.: 6110A).

### Total RNA Extraction and Reverse Transcription Polymerase Chain Reaction (RT-PCR)

Total RNA was isolated from the hybridoma cells following the technical manual of TRIzol® Reagent. The total RNA was analyzed by agarose gel electrophoresis.

Total RNA was reverse transcribed into cDNA using isotype-specific anti-sense primers or universal primers following the technical manual of PrimeScript™ 1st Strand cDNA Synthesis Kit. The antibody fragments of VH and VL were amplified according to the standard operating procedure of RACE of GenScript.

### Cloning of Antibody Gene, Screening and Sequencing

Amplified antibody fragments were separately cloned into a standard cloning vector using standard molecular cloning procedures.

Colony PCR screening was performed to identify clones with inserts of correct sizes. No less than five single colonies with inserts of correct sizes were sequenced for each antibody fragment.

### Results and Analysis

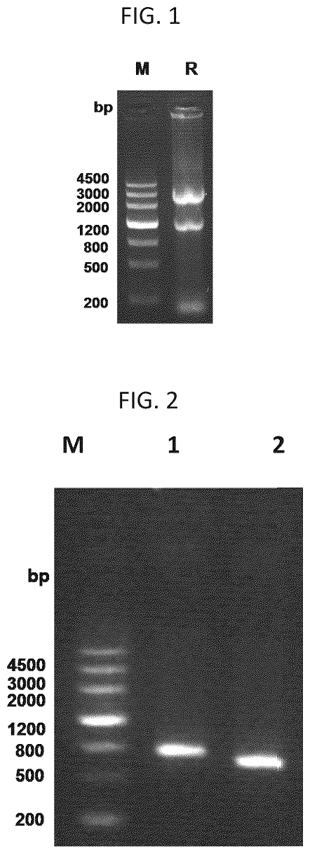

The isolated total RNA of the sample was run alongside a DNA marker, Marker III (TIANGEN, Cat. No.: MD103) on a 1.5% agarose/GelRed™ gel, as shown in FIG. 1.

Four microliters of PCR products of each sample were run alongside the DNA marker Marker III on a 1.5% agarose/GelRed™ gel, as shown in FIG. 2. The PCR products were purified and stored at −20° C. until further use.

### Sequencing Results and Analysis

Five single colonies with correct VH and VL insert sizes were sent for sequencing. The VH and VL genes of five different clones were found nearly identical. The DNA and amino acid sequences for the heavy chains, light chains and CDR regions are depicted in SEQ ID NOs: 1-16 (see Table 2), which correspond to anti-TL1A antibody 5C3D11.

TABLE 2
Nucleotide and Amino Acid sequencesfor 5C3D11

| | | | | | |
|---|---|---|---|---|---|
| | Sequence | | Leader | | |
| Antibody | Type | Size | Sequence | Sequence | SEQ ID No: |

5C3D11

| | | | | | |
|---|---|---|---|---|---|
| Heavy | DNA | 405 bp | FR1-CDR1- | ATGAAATGCAGCTGGGTTATCTTCTTCCTGATGGCAGTG | 1 |
| Chain | | | FR2-CDR2- | GTTACAGGGGTCAATTCAGAGGTTCAGCTGCAGCAGTCT | |
| | | | FR3-CDR3- | GGGGCAGAACTTGTGAAGCCAGGGGCCTCAGTCAAGTTG | |
| | | | FR4 | TCCTGCACAGCTTCTGGCTTCGACATTCAAGACACCTAT | |
| | | | | ATGCACTGGGTGAAGCAGAGGCCTGAACAGGGCCTGGAG | |
| | | | | TGGATTGGAAGGATTGATCCTGCGAGTGGACATACTAAA | |
| | | | | TATGACCCGAAGTTCCAGGTCAAGGCCACTATAACAACG | |
| | | | | GACACATCCTCCAACACAGCCTACCTGCAGCTCAGCAGC | |
| | | | | CTGACATCTGAGGACACTGCCGTCTATTACTGTTCTAGA | |
| | | | | TCGGGGGGCCTACCTGATGTCTGGGGCGCAGGGACCACG | |
| | | | | GTCACCGTCTCCTCA | |
| | | | CDR1 | GACACCTATATGCAC | 2 |
| | | | CDR2 | AGGATTGATCCTGCGAGTGGACATACTAAATATGACCCG | 3 |
| | | | | AAGTTCCAGGTC | |
| | | | CDR3 | TCGGGGGGCCTACCTGATGTC | 4 |
| Heavy | Amino | 135 AA | FR1-CDR1- | MKCSWVIFFLMAVVTGVNSEVQLQQSGAELVKPGASVKL | 5 |
| Chain | Acid | | FR2-CDR2- | SCTASGFDIQDTYMHWVKQRPEQGLEWIGRIDPASGHTK | |
| | | | FR3-CDR3- | YDPKFQVKATITTDTSSNTAYLQLSSLTSEDTAVYYCSR | |
| | | | FR4 | SGGLPDVWGAGTTVTVSS | |
| | | | CDR1 | DTYMH | 6 |
| | | | CDR2 | RIDPASGHTKYDPKFQV | 7 |
| | | | CDR3 | SGGLPDV | 8 |
| Light | DNA | 384 bp | FR1-CDR1- | ATGGATTTTCAAGTGCAGATTTTCAGCTTCCTGCTAATC | 9 |
| Chain | | | FR2-CDR2- | AGTGCTTCAGTCATAATGTCCAGAGGACAAATTGTTCTC | |
| | | | FR3-CDR3- | TCCCAGTCTCCTGCAATCCTGTCTGCATCTCCAGGGGAG | |
| | | | FR4 | AAGGTCACAATGACTTGCAGGGCCAGCTCAAGTGTAAGT | |
| | | | | TACATGTACTGGTACCAGCAGAAGCCTGGATCCTCCCCC | |
| | | | | AAACCCTGGATTTATGCCACATCCAACCTGGCTTCTGGA | |
| | | | | GTCCCTGATCGCTTCAGTGGCAGTGGGTCTGGGACCTCT | |
| | | | | TACTCTCTCACAATCAGCAGAGTGGAGGCTGAAGATGCT | |
| | | | | GCCACTTATTACTGCCAGCAGTGGAGTGGTAACCCACGG | |
| | | | | ACGTTCGGTGGAGGCACCAAGCTGGAAATCAAA | |
| | | | CDR1 | AGGGCCAGCTCAAGTGTAAGTTACATGTAC | 10 |
| | | | CDR2 | GCCACATCCAACCTGGCTTCT | 11 |
| | | | CDR3 | CAGCAGTGGAGTGGTAACCCACGGACG | 12 |
| Light | Amino | 128 AA | FR1-CDR1- | MDFQVQIFSFLLISASVIMSRGQIVLSQSPAILSASPGE | 13 |
| Chain | Acid | | FR2-CDR2- | KVIMTCRASSSVSYMYWYQQKPGSSPKPWIYATSNLASG | |
| | | | FR3-CDR3- | VPDRFSGSGSGTSYSLTISRVEAEDAATYYCQQWSGNPR | |
| | | | FR4 | TFGGGTKLEIK | |
| | | | CDR1 | RASSSVSYMY | 14 |
| | | | CDR2 | ATSNLAS | 15 |
| | | | CDR3 | QQWSGNPRT | 16 |

nt-nucleotide; aa-amino acid

### Monoclonal Antibody Sequencing of Hybridoma 9E12E5

Total RNA was extracted from frozen hybridoma cells and cDNA was synthesized from the RNA. PCR was then performed to amplify the variable regions (heavy and light chains) of the antibody, which were then cloned into a standard cloning vector separately and sequenced.

### Materials and Methods

Materials used include hybridoma cells; TRIzol® Reagent (Ambion, Cat. No.: 15596-026); and PrimeScript™ 1st Strand cDNA Synthesis Kit (Takara, Cat. No.: 6110A).

### Total RNA Extraction and Reverse Transcription Polymerase Chain Reaction (RT-PCR)

Total RNA was isolated from the hybridoma cells following the technical manual of TRIzol® Reagent. The total RNA was analyzed by agarose gel electrophoresis.

Total RNA was reverse transcribed into cDNA using isotype-specific anti-sense primers or universal primers following the technical manual of PrimeScript™ 1st Strand cDNA Synthesis Kit. The antibody fragments of VH and VL were amplified according to the standard operating procedure of RACE of GenScript.

### Cloning of Antibody Genes, Screening and Sequencing

Amplified antibody fragments were separately cloned into a standard cloning vector using standard molecular cloning procedures.

Colony PCR screening was performed to identify clones with inserts of correct sizes. No less than five single colonies with inserts of correct sizes were sequenced for each antibody fragment.

### Results and Analysis

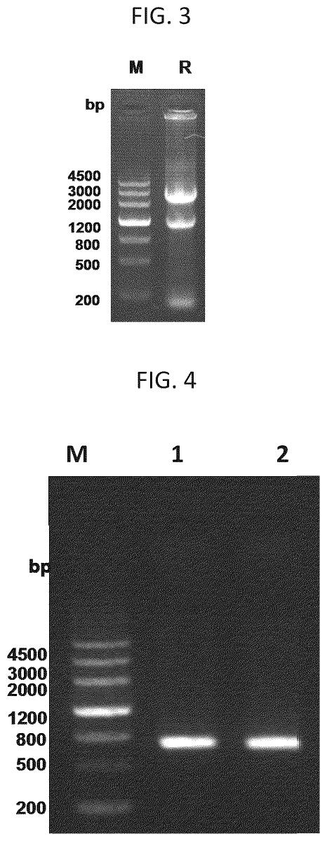

The isolated total RNA of the sample was run alongside a DNA marker Marker III (TIANGEN, Cat. No.: MD103) on a 1.5% agarose/GelRed™ gel, as shown in FIG. 3.

Four microliters of PCR products of each sample were run alongside the DNA marker Marker III on a 1.5% agarose/GelRed™ gel, as shown in FIG. 4. The PCR products were purified and stored at −20° C. until further use.

### Sequencing Results and Analysis

Five single colonies with correct VH and VL insert sizes were sent for sequencing. The VH and VL genes of five different clones were found nearly identical. The DNA and amino acid sequences for the heavy chains, light chains and CDR regions are depicted in SEQ ID NOs: 17-32 (see Table 3), which correspond to anti-TL1A antibody 9E12E5.

TABLE 3
Nucleotide and Amino Acid sequencesfor 9E12E5

| | | | | | |
|---|---|---|---|---|---|
| | Sequence | | Leader | | |
| Antibody | Type | Size | Sequence | Sequence | SEQ ID No: |

9E12E5

| | | | | | |
|---|---|---|---|---|---|
| Heavy | DNA | 405 bp | FR1-CDR1- | ATGGGATGGAGCTGGGTCTTTCTCTTCCTCCTGTCAGTGAC | 17 |
| Chain | | | FR2-CDR2- | TGCAGGTGTCCACTCCCAGGTTCACCTGCAGCAGTCTGGAC | |
| | | | FR3-CDR3- | CTGAACTGGTAAAGCCTGGGGCTTCAGTGAAGTTGTCCTGC | |
| | | | FR4 | AAGGCTTCTGGCTACACCTTCACAAAGTATGATATAAACTG | |
| | | | | GGTGAGGCAGAGGCCTGAACAGGGACTTGAGTGGATTGGAT | |
| | | | | GGATTTTTCCTGGAGATGGTAGAACTGACTACAATGAGAAG | |
| | | | | TTCAAGGGTAAGGCCACACTGACTACAGACAAATCCTCCAG | |
| | | | | CACAGCCTACATGGAGGTCAGCAGGCTGACATCTGAGGACT | |
| | | | | CTGCTGTCTATTTCTGTGCAAGATATGGCCCCGCTATGGAC | |
| | | | | TACTGGGGTCAAGGAACCTCAGTCACCGTCGCCTCA | |
| | | | CDR1 | AAGTATGATATAAAC | 18 |
| | | | CDR2 | TGGATTTTTCCTGGAGATGGTAGAACTGACTACAATGAGAA | 19 |
| | | | | GTTCAAGGGT | |
| | | | CDR3 | TATGGCCCCGCTATGGACTA | 20 |
| Heavy | Amino | 135 AA | FR1-CDR1- | MGWSWVFLFLLSVTAGVHSQVHLQQSGPELVKPGASVKLSC | 21 |
| Chain | Acid | | FR2-CDR2- | KASGYTFTKYDINWVRQRPEQGLEWIGWIFPGDGRTDYNEK | |
| | | | FR3-CDR3- | FKGKATLTTDKSSSTAYMEVSRLTSEDSAVYFCARYGPAMD | |
| | | | FR4 | YWGQGTSVTVAS | |
| | | | CDR1 | KYDIN | 22 |
| | | | CDR2 | WIFPGDGRTDYNEKFKG | 23 |
| | | | CDR3 | YGPAMDY | 24 |
| Light | DNA | 393 bp | FR1-CDR1- | ATGAAGTTGCCTGTTAGGCTGTTGGTGCTGATGTTCTGGAT | 25 |
| Chain | | | FR2-CDR2- | TCCTGCTTCCAGCAGTGATGTTTTGATGACCCAAACTCCAC | |
| | | | FR3-CDR3- | TCTCCCTGCCTGTCAGTCTTGGAGATCAAGCCTCCATCTCT | |
| | | | FR4 | TGCAGATCTAGTCAGACCATTGTACATAGTAATGGAGACAC | |
| | | | | CTATTTAGACTGGTTCCTGCAGAAACCAGGCCAGTCTCCAA | |
| | | | | AGCTCCTGATCTACAAAGTTTCCAACCGATTTTCTGGGGTC | |
| | | | | CCAGACAGGTTCAGTGGCAGTGGATCAGGGACAGATTTCAC | |
| | | | | ACTCAAGATCAGCAGAGTGGAGGCTGAGGATCTGGGAGTTT | |
| | | | | ATTACTGCTTTCAAGGTTCACATGTTCCGTACACGTTCGGA | |
| | | | | GGGGGGACCAAGCTGGAAATAAAA | |
| | | | CDR1 | AGATCTAGTCAGACCATTGTACATAGTAATGGAGACACCTA | 26 |
| | | | | TTTAGAC | |
| | | | CDR2 | AAAGTTTCCAACCGATTTTCT | 27 |
| | | | CDR3 | TTTCAAGGTTCACATGTTCCGTACACG | 28 |
| Light | Amino | 131 AA | FR1-CDR1- | MKLPVRLLVLMFWIPASSSDVLMTQTPLSLPVSLGDQASIS | 29 |
| Chain | Acid | | FR2-CDR2- | CRSSQTIVHSNGDTYLDWFLQKPGQSPKLLIYKVSNRFSGV | |
| | | | FR3-CDR3- | PDRESGSGSGTDFTLKISRVEAEDLGVYYCFQGSHVPYTFG | |
| | | | FR4 | GGTKLEIK | |
| | | | CDR1 | RSSQTIVHSNGDTYLD | 30 |
| | | | CDR2 | KVSNRFS | 31 |
| | | | CDR3 | FQGSHVPYT | 32 |

nt-nucleotide; aa-amino acid

### Example 2

Alignment comparison of the two antibody sequences using BLASTP 2.2.32+ for the heavy chain variable region (Table 4) and the light chain variable region (Table 5).

TABLE 4
BLAST Analysis of the Heavy Chain Variable Region

Leader sequence-FR1-CDR1-FR2-CDR2-FR3-CDR3-FR4 (SEQ ID NOS 46-47 disclosed,
respectively, in order of appearance)
Query 1 = 9E12E5
Subject = 5C3D11
Length = 135
Score = 173 bits (439), Expect = 1e-60, Method: Compositional matrix adjust.
Identities = 84/135 (62%), Positives = 104/135 (77%), Gaps = 0/135 (0%)

| | | | |
|---|---|---|---|
| Query | 1 | MGWSWVFLFLLSVTAGVHSQVHLQQSGPELVKPGASVKLSCKASGYTFTKYDINWVRQRP | 60 |
| | | M SWV FL++V GV+S+V LQQSG ELVKPGASVKLSC ASG+ ++WV+QRP | |
| Sbjct | 1 | MKCSWVIFFLMAVVTGVNSEVQLQQSGAELVKPGASVKLSCTASGFDIQDTYMHWVKQRP | 60 |
| Query | 61 | EQGLEWIGWIFPGDGRTDYNEKFKGKATLTTDKSSSTAYMEVSRLTSEDSAVYFCARYGP | 120 |
| | | EQGLEWIG I P G T Y+ KF+ KAT+TTD SS+TAY+++S LTSED+AVY+C+R G | |
| Sbjct | 61 | EQGLEWIGRIDPASGHTKYDPKFQVKATITTDTSSNTAYLQLSSLTSEDTAVYYCSRSGG | 120 |
| Query | 121 | AMDYWGQGTSVTVAS | 135 |
| | | D WG GT+VTV+S | |
| Sbjct | 121 | LPDVWGAGTTVTVSS | 135 |

TABLE 5
BLAST Analysis of the Light Chain Variable Region

Leader sequence-FR1-CDR1-FR2-CDR2-FR3-CDR3-FR4 (SEQ ID NOS 48-49 disclosed,
respectively, in order of appearance)
Query 1 = 9E12E5
Subject = 5C3D11
Length = 128
Score = 107 bits (268), Expect = 4e-35, Method: Compositional matrix adjust.
Identities = 62/115 (54%), Positives = 80/115 (70%), Gaps = 6/115 (5%)

| | | | |
|---|---|---|---|
| Query | 17 | SSSDVLMTQTPLSLPVSLGDQASISCRSSQTIVHSNGDTYLDWFLQKPGQSPKLLIYKVS | 76 |
| | | S ++++Q+P L S G++ +++CR+S ++ +Y+ W+ QKPG SPK IY S | |
| Sbjct | 20 | SRGQIVLSQSPAILSASPGEKVTMTCRASSSV------SYMYWYQQKPGSSPKPWIYATS | 73 |
| Query | 77 | NRFSGVPDRFSGSGSGTDFTLKISRVEAEDLGVYYCFQGSHVPYTFGGGTKLEIK | 131 |
| | | N SGVPDRFSGSGSGT ++L ISRVEAED YYC Q S P TFGGGTKLEIK | |
| Sbjct | 74 | NLASGVPDRFSGSGSGTSYSLTISRVEAEDAATYYCQQWSGNPRTFGGGTKLEIK | 128 |

### Example 3

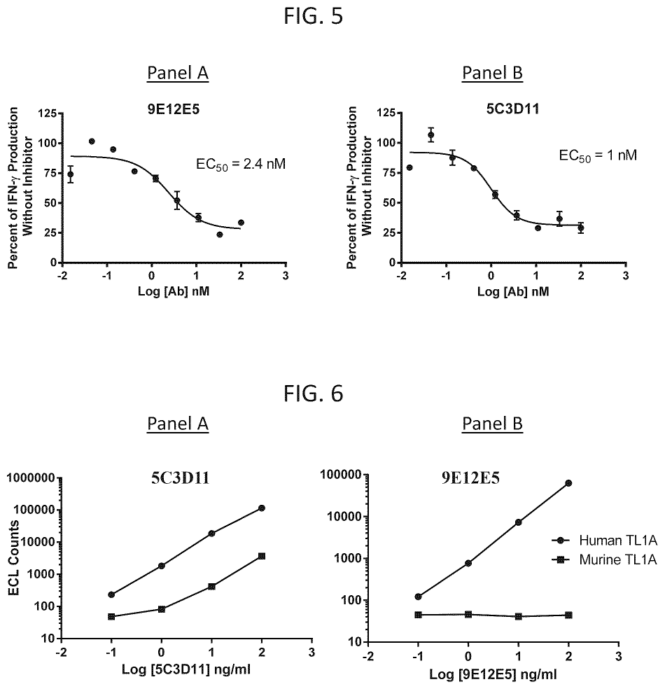

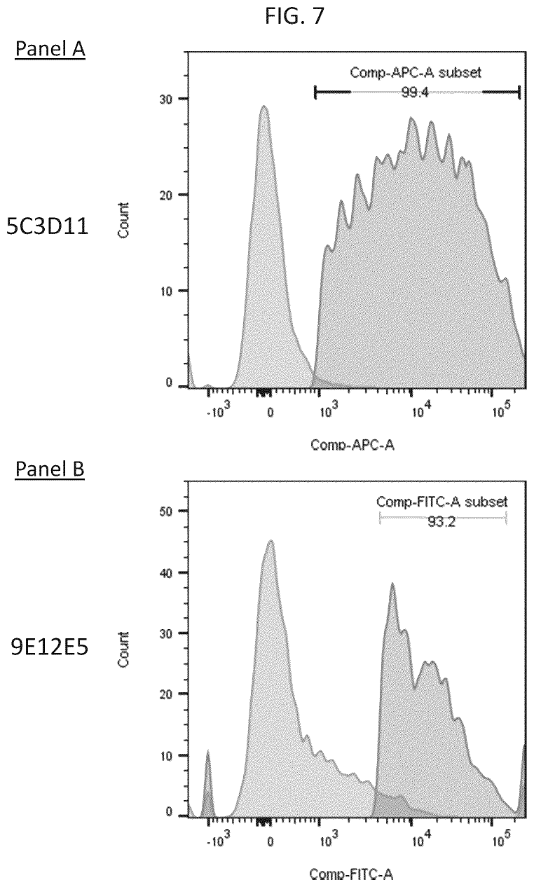

Anti-human TL1A monoclonal antibodies 9E12E5 and 5C3D11 neutralize the activity of human TL1A in vitro. The effect of the TL1A antibodies on IFN-γ production was assessed and both TL1A antibodies demonstrate an inhibition of TL1 A induced IFN-γ production, as shown in FIG. 5. MSD plates were coated with murine TL1A and incubated with various concentrations of 5C3D11 or 9E12E5 to evaluate the ability of the antibody to recognize murine TL1A, as shown in FIG. 6. Human TL1A was included as a positive control. 5C3D11 recognizes murine TL1A in a concentration dependent manner. The binding profile of both the 5C3D11 and 9E12E5 antibodies were assessed using a TL1A transfected HEK293 cell line and compared to the untransfected parental HEK293 cell line. Fluorescent staining of the anti-TL1A antibodies in TL1A expressing HEK293 cells is compared to the untransfected parental HEK293 cells, as shown in FIG. 7. The binding affinity for the anti-TL1A antibodies was measured by Biacore, with the dissociation constants shown in Table 6.

TABLE 6
Binding Affinity Values for anti-TL1A Antibodies

| Antibody | Analyte | K_{D} (M) |
|---|---|---|
| 5C3D11 | TL1A | 7.90E−11 |
| 9E12E5 | TL1A | 5.20E−10 |

### Example 4

The efficacy of anti-TL1A antibodies in animal models of colitis is performed.

Anti-TL1A antibodies are tested in rodent models of acute colitis induced by intrarectal administration of di- or tri-nitrobenzenesulfonic acid (D/TNBS) or oxazolone, and chronic colitis induced by administration of DSS in drinking water or transfer of CD45RB^{hi} T cells. DNBS and oxazolone induce localized ulceration and inflammation. DSS administration induces robust generalized inflammation of the intestinal tract characterized by erosive lesions and inflammatory infiltrate. Symptoms of all these models usually include diarrhea, occult blood, weight loss and occasionally rectal prolapse.

In a prophylactic model, antibody treatment begins at the start of administration of the colitis-inducing compound. In a therapeutic model, antibody treatment begins several days after commencement of induction. The effect of the treatment on weight, stool consistency and occult blood, as well as microscopic effects on epithelial integrity and degree of inflammatory infiltrate is determined. Daily clinical scoring is performed based on stool consistency and presence of occult blood giving a disease activity index (DAI) score.

### Example 5

A phase 1 clinical trial is performed to evaluate the safety, tolerability, pharmacokinetics and pharmacodynamics of an anti-TL1A antibody provided herein in subjects having Crohn's Disease.

Single ascending dose (SAD) arms: Subjects in each group (subjects are grouped based on the presence or absence of a risk variant) receive either a single dose of the antibody or a placebo. Exemplary doses are 1, 3, 10, 30, 100, 300, 600 and 800 mg of antibody. Safety monitoring and PK assessments are performed for a predetermined time. Based on evaluation of the PK data, and if the antibody is deemed to be well tolerated, dose escalation occurs, either within the same groups or a further group of healthy subjects. Dose escalation continues until the maximum dose has been attained unless predefined maximum exposure is reached or intolerable side effects become apparent.

Multiple ascending dose (MAD) arms: Subjects in each group (subjects are grouped based on the presence or absence of a risk variant) receive multiple doses of the antibody or a placebo. The dose levels and dosing intervals are selected as those that are predicted to be safe from the SAD data. Dose levels and dosing frequency are chosen to achieve therapeutic drug levels within the systemic circulation that are maintained at steady state for several days to allow appropriate safety parameters to be monitored. Samples are collected and analyzed to determination PK profiles.

Inclusion Criteria: Healthy subjects of non-childbearing potential between the ages of 18 and 55 years. Healthy is defined as no clinically relevant abnormalities identified by a detailed medical history, full physical examination, including blood pressure and pulse rate measurement, 12 lead ECG and clinical laboratory tests. Female subjects of non-childbearing potential must meet at least one of the following criteria: (1) achieved postmenopausal status, defined as: cessation of regular menses for at least 12 consecutive months with no alternative pathological or physiological cause; and have a serum follicle stimulating hormone (FSH) level within the laboratory's reference range for postmenopausal females; (2) have undergone a documented hysterectomy and/or bilateral oophorectomy; (3) have medically confirmed ovarian failure. All other female subjects (including females with tubal ligations and females that do NOT have a documented hysterectomy, bilateral oophorectomy and/or ovarian failure) will be considered to be of childbearing potential. Body Mass Index (BMI) of 17.5 to 30.5 kg/m²; and a total body weight>50 kg (110 lbs). Evidence of a personally signed and dated informed consent document indicating that the subject (or a legal representative) has been informed of all pertinent aspects of the study.

Two groups of healthy subjects are selected: subjects having a risk variant whose presence is associated with an increase in susceptibility to Crohn's Disease, and subjects lacking the risk variant.

Exclusion Criteria: Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing). Subjects with a history of or current positive results for any of the following serological tests: Hepatitis B surface antigen (HBsAg), Hepatitis B core antibody (HBcAb), anti-Hepatitis C antibody (HCV Ab) or human immunodeficiency virus (HIV). Subjects with a history of allergic or anaphylactic reaction to a therapeutic drug. Treatment with an investigational drug within 30 days (or as determined by the local requirement, whichever is longer) or 5 half-lives or 180 days for biologics preceding the first dose of study medication. Pregnant females; breastfeeding females; and females of childbearing potential.

Primary Outcome Measures: Incidence of dose limiting or intolerability treatment related adverse events (AEs)[Time Frame: 12 weeks]. Incidence, severity and causal relationship of treatment emergent AEs (TEAEs) and withdrawals due to treatment emergent adverse events [Time Frame: 12 weeks]. Incidence and magnitude of abnormal laboratory findings [Time Frame: 12 weeks]. Abnormal and clinically relevant changes in vital signs, blood pressure (BP) and electrocardiogram (ECG) parameters [Time Frame: 12 weeks].

### Secondary Outcome Measures:

Single Ascending Dose: Maximum Observed Plasma Concentration (Cmax) [Time Frame: 12 weeks]. Single Ascending Dose: Time to Reach Maximum Observed Plasma Concentration (Tmax)[Time Frame: 12 weeks]. Single Ascending Dose: Area under the plasma concentration-time profile from time zero to 14 days (AUC14 days)[Time Frame: 12 weeks]. Single Ascending Dose: Area under the plasma concentration-time profile from time zero extrapolated to infinite time (AUCinf)[Time Frame: 12 weeks]. Single Ascending Dose: Area under the plasma concentration-time profile from time zero to the time of last quantifiable concentration (AUClast)[Time Frame: 12 weeks]. Single Ascending Dose: Dose normalized maximum plasma concentration (Cmax[dn])[Time Frame: 12 weeks]. Single Ascending Dose: Dose normalized area under the plasma concentration-time profile from time zero extrapolated to infinite time (AUCinf[dn])[Time Frame: 12 weeks]. Single Ascending Dose: Dose normalized area under the plasma concentration-time profile from time zero to the time of last quantifiable concentration (AUClast[dn])[Time Frame: 12 weeks]. Single Ascending Dose: Plasma Decay Half-Life (t½)[Time Frame: 12 weeks]. Plasma decay half-life is the time measured for the plasma concentration to decrease by one half. Single Ascending Dose: Mean residence time (MRT)[Time Frame: 12 weeks]. Single Ascending Dose: Volume of Distribution at Steady State (Vss)[Time Frame: 6 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Single Ascending Dose: Systemic Clearance (CL) [Time Frame: 6]. CL is a quantitative measure of the rate at which a drug substance is removed from the body.

Multiple Ascending Dose First Dose: Maximum Observed Plasma Concentration (Cmax)[Time Frame: 12 weeks]. Multiple Ascending Dose First Dose: Time to Reach Maximum Observed Plasma Concentration (Tmax)[Time Frame: 12 weeks]. Multiple Ascending Dose First Dose: Area under the plasma concentration-time profile from time zero to time t, the dosing interval where τ=2 weeks (AUCt)[Time Frame: 12 weeks]. Multiple Ascending Dose First Dose: Dose normalized maximum plasma concentration (Cmax[dn]) [Time Frame: 12 weeks]. Multiple Ascending Dose First Dose: Dose normalized Area under the plasma concentration-time profile from time zero to time t, the dosing interval where t=2 weeks (AUCt[dn])[Time Frame: 12 weeks]. Plasma Decay Half-Life (t½)[Time Frame: 12 weeks]. Plasma decay half-life is the time measured for the plasma concentration to decrease by one half. Multiple Ascending Dose First Dose: Mean residence time (MRT)[Time Frame: 12 weeks]. Apparent Volume of Distribution (Vz/F)[Time Frame: 12 weeks]. Volume of distribution is defined as the theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose First Dose: Volume of Distribution at Steady State (Vss)[Time Frame: 12 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Multiple Ascending Dose First Dose: Apparent Oral Clearance (CL/F)[Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance is estimated from population pharmacokinetic (PK) modeling. Drug clearance is a quantitative measure of the rate at which a drug substance is removed from the blood. Multiple Ascending Dose First Dose: Systemic Clearance (CL)[Time Frame: 12 weeks]. CL is a quantitative measure of the rate at which a drug substance is removed from the body.

Multiple Ascending Dose Multiple Dose: Maximum Observed Plasma Concentration (Cmax)[Time Frame: 12 weeks]. Multiple Ascending Dose Multiple Dose: Time to Reach Maximum Observed Plasma Concentration (Tmax)[Time Frame: 12 weeks]. Multiple Ascending Dose Multiple Dose: Area under the plasma concentration-time profile from time zero to time t, the dosing interval where t=2 weeks (AUCt)[Time Frame: 12 weeks]. Multiple Ascending Dose Multiple Dose: Dose normalized maximum plasma concentration (Cmax[dn]) [Time Frame: 12 weeks]. Multiple Ascending Dose Multiple Dose: Dose normalized Area under the plasma concentration-time profile from time zero to time t, the dosing interval where t=2 weeks (AUCt[dn])[Time Frame: 12 weeks]. Multiple Ascending Dose Multiple Dose: Plasma Decay Half-Life (t½)[Time Frame: 12 weeks]. Plasma decay half-life is the time measured for the plasma concentration to decrease by one half. Multiple Ascending Dose Multiple Dose: Apparent Volume of Distribution (Vz/F)[Time Frame: 12 weeks]. Volume of distribution is defined as the theoretical volume in which the total amount of drug would need to be uniformly distributed to produce the desired plasma concentration of a drug. Apparent volume of distribution after oral dose (Vz/F) is influenced by the fraction absorbed. Multiple Ascending Dose Multiple Dose: Volume of Distribution at Steady State (Vss)[Time Frame: 12 weeks]. Volume of distribution is defined as the theoretical volume in which the total amount of drug would need to be uniformly distributed to produce the desired blood concentration of a drug. Steady state volume of distribution (Vss) is the apparent volume of distribution at steady-state. Multiple Ascending Dose Multiple Dose: Apparent Oral Clearance (CL/F)[Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance was estimated from population pharmacokinetic (PK) modeling. Drug clearance is a quantitative measure of the rate at which a drug substance is removed from the blood. Multiple Ascending Dose Multiple Dose: Systemic Clearance (CL)[Time Frame: 12 weeks]. CL is a quantitative measure of the rate at which a drug substance is removed from the body. Multiple Ascending Dose Multiple Dose: Minimum Observed Plasma Trough Concentration (Cmin)[Time Frame: 12 weeks]. Multiple Ascending Dose Multiple Dose: Average concentration at steady state (Cav)[Time Frame: 12 weeks]. Multiple Ascending Dose Multiple Dose: Observed accumulation ratio (Rac)[Time Frame: 12 weeks]. Multiple Ascending Dose Multiple Dose: Peak to trough fluctuation (PTF) [Time Frame: 12 weeks]. Multiple Ascending Dose Additional Parameter: estimate of bioavailability (F) for subcutaneous administration at the corresponding intravenous dose [Time Frame: 12 weeks]. Immunogenicity for both Single Ascending Dose and Multiple Ascending Dose: Development of anti-drug antibodies (ADA)[Time Frame: 12 weeks].

### Example 6

A phase 1b open label clinical trial is performed to evaluate efficacy of an anti-TL1A antibody provided herein on patients having a risk variant associated with Crohn's Disease.

Arms: 10 patients positive for a risk variant whose presence is associated with an increase in susceptibility to Crohn's Disease are administered the antibody. 5-10 patients negative for the risk variant are administered the antibody. Patients are monitored in real-time. Central ready of endoscopy and biopsy is employed, with readers blinded to point of time of treatment and endpoints.

Inclusion Criteria: Two groups of subjects are selected: subjects having a risk variant whose presence is associated with an increase in susceptibility to Crohn's Disease, and subjects lacking the risk variant.

Primary Outcome Measures: Simple Endoscopic Score for Crohn's Disease (SESCD), Crohn's Disease Activity Index (CDAI), and Patient Reported Outcome (PRO). If risk variant positive group shows 50% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 7

A phase 2a clinical trial is performed to evaluate the efficacy of an anti-TL1A antibody provided herein in subjects having Crohn's Disease.

Arms: 40 patients per arm (antibody and placebo arms) are treated with antibody or placebo for 12 weeks. An interim analysis is performed after 20 patients from each group are treated at the highest dose to look for a 40-50% delta between placebo and treated group in primary outcome (50% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 8

Humanized anti-TL1A antibodies were generated. Briefly, a library of antibodies was created comprising the CDRs of 5C3D11 and framework regions from human germline antibodies. The library was screened using phage display to identify antibodies having affinity to human TL1A (hTL1A) antigen. Sixty clones were affinity ranked using surface plasmon resonance (SPR). Five antibodies were selected based on affinity data and evaluation of framework sequence: AS12816, AS12819, AS12823, AS12824, and AS12825. Sequence data for the selected clones is shown in Table 7 (VH) and Table 8 (VL). Affinity data for the five selected clones is shown in Table 9. Four of the five selected clones (AS12816, AS12819, AS12823, and AS12824) were selected for multi-cycle affinity measurement, with corresponding data shown in Table 10.

TABLE 7
Humanized anti-TL1A clones: heavy chain sequences.

| | SEQ | |
|---|---|---|
| Clone | ID NO. | Sequence |
| AS12824 | 35 | QVQLVQSGAEVKKPGASVKVSCKASGFDICDTYMH |
| | | WVKQRPGQGLEWIGRIDPASGHTKYDPKFQVRATI |
| | | TTDTSTSTAYLELSSLRSEDTAVYYCARSGGLPDV |
| | | WGQGTTVTVSS |
| AS12823 | 36 | QVQLVQSGAEVKKPGASVKLSCKASGFDICDTYMH |
| | | WVRQRPGQGLEWIGRIDPASGHTKYDPKFQVRATM |
| | | TTDTSTSTVYLELSSLRSEDTAVYYCARSGGLPDV |
| | | WGQGTTVTVSS |
| AS12819 | 37 | QVQLVQSGAEVVKPGASVKLSCKASGFDICDTYMH |
| | | WVRQRPGQGLEWMGRIDPASGHTKYDPKFQVRVTM |
| | | TTDTSTSTVYLELSSLRSEDTAVYYCARSGGLPDV |
| | | WGQGTTVTVSS |
| AS12816 | 38 | QVQLVQSGAEVKKPGASVKVSCKASGFDICDTYMH |
| | | WVKQRPGQGLEWIGRIDPASGHTKYDPKFQVRATI |
| | | TRDTSTSTAYLELSSLRSEDTAVYYCSRSGGLPDV |
| | | WGQGTTVTVSS |
| AS12825 | 39 | QVQLVQSGAEVKKPGASVKVSCKASGFDICDTYMH |
| | | WVKQAPGQGLEWMGRIDPASGHTKYDPKFQVRATM |
| | | TTDTSTSTAYLELSSLRSEDTAVYYCSRSGGLPDV |
| | | WGQGTTVTVSS |

TABLE 8
Humanized anti-TL1A clones: light chain sequences.

| | SEQ | |
|---|---|---|
| Clone | ID NO. | Sequence |
| AS12824 | 40 | EIVLTQSPGTLSASPGERATMSCRASSSVSYMYWY |
| | | QQKPGQAPRPWIYATSNLASGVPDRFSGSGSGTDY |
| | | TLTISRVEPEDFAVYYCQQWSGNPRTFGGGTKLEI |
| | | K |
| AS12823 | 41 | EIVLTQSPGTLSLSPGERATMSCRASSSVSYMYWY |
| | | QQKPGQAPRPWIYATSNLASGIPDRFSGSGSGTDF |
| | | TLTISRVEPEDFAVYYCQQWSGNPRTFGGGTKLEI |
| | | K |
| AS12819 | 42 | EIVLTQSPGTLSLSPGERVTMSCRASSSVSYMYWY |
| | | QQKPGQAPRPWIYATSNLASGVPDRFSGSGSGTDF |
| | | TLTISRVEPEDFAVYYCQQWSGNPRTFGGGTKVEI |
| | | K |
| AS12816 | 43 | EIVLTQSPGTLSASPGERVTLSCRASSSVSYMYWY |
| | | QQKPGQAPRPWIYATSNLASGVPDRFSGSGSGTDF |
| | | TLTISRLEPEDFAVYYCQQWSGNPRTFGGGTKLEI |
| | | K |
| AS12825 | 44 | EIVLTQSPGTLSASPGERVTMSCRASSSVSYMYWY |
| | | QQKPGQAPRLLIYATSNLASGVPDRFSGSGSGTDF |
| | | TLTISRVEPEDFAVYYCQQWSGNPRTFGGGTKLEI |
| | | K |

TABLE 9
Humanized anti-TL1A clones: light chain mutations.

| | | | | | | | |
|---|---|---|---|---|---|---|---|
| | | | ka | | | Rmax | Chi2 |
| Clone NO. | Ligand | Curve | (1/Ms) | kd (1/s) | KD (M) | (RU) | (RU2) |

Controls

| | | | | | | |
|---|---|---|---|---|---|---|
| graft-BM- | Fc = 2-1 | 1.01E+06 | 6.39E−06 | 6.32E−12 | 445.9 | 1.48 |
| SH | | | | | | |
| graft-BM- | Fc = 2-1 | 1.46E+06 | 1.70E−05 | 1.16E−11 | 437.2 | 49.10 |
| SH | | | | | | |
| chimeric | Fc = 4-1 | 1.56E+06 | 3.70E−05 | 2.37E−11 | 247.9 | 44.00 |
| chimeric | Fc = 4-1 | 9.03E+05 | 3.12E−05 | 3.46E−11 | 269.9 | 1.00 |

Affinity ranked clones

| | | | | | | | |
|---|---|---|---|---|---|---|---|
| AS12824 | 15 | Fc = 3-1 | 1.03E+06 | 1.69E−05 | 1.63E−11 | 324 | 1.09 |
| AS12835 | 28 | Fc = 2-1 | 9.22E+05 | 2.73E−05 | 2.96E−11 | 119.9 | 25.70 |
| AS12823 | 14 | Fc = 3-1 | 8.64E+05 | 3.66E−05 | 4.24E−11 | 155 | 0.58 |
| AS12819 | 10 | Fc = 3-1 | 8.25E+05 | 3.81E−05 | 4.62E−11 | 178 | 1.05 |
| AS12831 | 23 | Fc = 4-1 | 8.25E+05 | 3.83E−05 | 4.64E−11 | 167.4 | 0.33 |
| AS12821 | 12 | Fc = 3-1 | 1.05E+06 | 4.93E−05 | 4.72E−11 | 342 | 1.11 |
| AS12851 | 54 | Fc = 4-1 | 8.78E+05 | 4.52E−05 | 5.15E−11 | 185.8 | 0.82 |
| AS12816 | 6 | Fc = 2-1 | 7.63E+05 | 4.73E−05 | 6.20E−11 | 273.4 | 0.39 |
| AS12850 | 52 | Fc = 2-1 | 1.37E+06 | 9.21E−05 | 6.71E−11 | 97.5 | 1.71 |
| AS12817 | 7 | Fc = 2-1 | 1.00E+06 | 6.92E−05 | 6.89E−11 | 93.5 | 0.37 |
| AS12839 | 33 | Fc = 3-1 | 9.93E+05 | 7.46E−05 | 7.52E−11 | 97.7 | 0.62 |
| AS12837 | 30 | Fc = 2-1 | 8.80E+05 | 6.64E−05 | 7.55E−11 | 195.4 | 0.41 |
| AS12843 | 38 | Fc = 3-1 | 9.22E+05 | 7.10E−05 | 7.70E−11 | 113.9 | 0.47 |
| AS12838 | 31 | Fc = 2-1 | 9.04E+05 | 7.14E−05 | 7.90E−11 | 112.7 | 0.24 |
| AS12828 | 20 | Fc = 4-1 | 7.31E+05 | 5.80E−05 | 7.94E−11 | 180.6 | 0.48 |
| AS12842 | 37 | Fc = 3-1 | 1.10E+06 | 9.07E−05 | 8.23E−11 | 244.7 | 2.15 |
| AS12841 | 35 | Fc = 3-1 | 8.32E+05 | 6.87E−05 | 8.26E−11 | 187.9 | 0.54 |
| AS12822 | 13 | Fc = 3-1 | 8.95E+05 | 7.46E−05 | 8.33E−11 | 121.8 | 0.82 |
| AS12815 | 4 | Fc = 2-1 | 8.25E+05 | 6.91E−05 | 8.37E−11 | 197.5 | 0.40 |
| AS12820 | 11 | Fc = 3-1 | 1.14E+06 | 9.54E−05 | 8.40E−11 | 78.1 | 0.54 |
| AS12833 | 26 | Fc = 2-1 | 7.22E+05 | 6.66E−05 | 9.22E−11 | 160.9 | 2.91 |
| AS12818 | 8 | Fc = 2-1 | 8.61E+05 | 8.64E−05 | 1.00E−10 | 142.1 | 0.35 |
| AS12825 | 16 | Fc = 3-1 | 5.18E+05 | 5.45E−05 | 1.05E−10 | 132.1 | 2.95 |
| AS12848 | 49 | Fc = 2-1 | 7.95E+05 | 8.47E−05 | 1.06E−10 | 125.7 | 0.74 |
| AS12813 | 2 | Fc = 2-1 | 7.03E+05 | 8.20E−05 | 1.17E−10 | 136 | 0.36 |
| AS12845 | 41 | Fc = 4-1 | 7.65E+05 | 9.35E−05 | 1.22E−10 | 99 | 0.37 |
| AS12849 | 50 | Fc = 2-1 | 8.97E+05 | 1.18E−04 | 1.32E−10 | 92.3 | 0.69 |
| AS12846 | 42 | Fc = 4-1 | 7.80E+05 | 1.03E−04 | 1.33E−10 | 112.2 | 0.34 |
| AS12847 | 45 | Fc = 4-1 | 8.30E+05 | 1.11E−04 | 1.34E−10 | 197.6 | 1.11 |
| AS12832 | 24 | Fc = 4-1 | 7.31E+05 | 9.90E−05 | 1.36E−10 | 104.6 | 0.29 |
| AS12834 | 27 | Fc = 2-1 | 8.38E+05 | 1.31E−04 | 1.57E−10 | 111.3 | 0.58 |
| AS12830 | 22 | Fc = 4-1 | 4.48E+05 | 7.86E−05 | 1.76E−10 | 282.7 | 1.07 |
| AS12840 | 34 | Fc = 3-1 | 9.04E+05 | 1.62E−04 | 1.79E−10 | 37.6 | 0.45 |
| AS12852 | 56 | Fc = 4-1 | 5.06E+05 | 9.18E−05 | 1.81E−10 | 113.6 | 1.65 |
| AS12836 | 29 | Fc = 2-1 | 8.85E+05 | 1.61E−04 | 1.82E−10 | 120.2 | 1.14 |
| AS12827 | 19 | Fc = 4-1 | 3.15E+05 | 5.83E−05 | 1.85E−10 | 308.3 | 1.45 |
| AS12826 | 18 | Fc = 4-1 | 4.91E+05 | 9.15E−05 | 1.86E−10 | 116.9 | 2.33 |
| AS12814 | 3 | Fc = 2-1 | 4.56E+05 | 9.64E−05 | 2.11E−10 | 102.2 | 2.51 |
| AS12829 | 21 | Fc = 4-1 | 3.48E+05 | 1.48E−04 | 4.24E−10 | 202.9 | 0.54 |
| AS12844 | 39 | Fc = 3-1 | 4.95E+05 | 2.27E−04 | 4.58E−10 | 60.7 | 0.54 |
| AS12853 | 59 | Fc = 3-1 | 5.11E+05 | 3.17E−04 | 6.21E−10 | 104 | 2.26 |
| | 32 | Fc = 2-1 | 1.01E+06 | 3.26E−05 | 3.24E−11 | 90.6 | 0.983 |
| | 40 | Fc = 3-1 | 1.08E+06 | 4.27E−05 | 3.97E−11 | 79.2 | 1.06 |
| | 36 | Fc = 3-1 | 5.46E+05 | 2.63E−05 | 4.82E−11 | 59.1 | 28.1 |
| | 44 | Fc = 4-1 | 8.64E+05 | 4.19E−05 | 4.85E−11 | 76.6 | 28.1 |
| | 58 | Fc = 3-1 | 1.12E+06 | 7.72E−05 | 6.91E−11 | 86.6 | 0.616 |
| | 48 | Fc = 4-1 | 9.86E+05 | 7.46E−05 | 7.56E−11 | 56 | 0.474 |
| | 57 | Fc = 3-1 | 1.05E+06 | 8.69E−05 | 8.29E−11 | 48.2 | 0.825 |
| | 60 | Fc = 3-1 | 2.02E+06 | 1.78E−04 | 8.81E−11 | 53.9 | 1.77 |
| | 25 | Fc = 2-1 | 1.03E+06 | 9.75E−05 | 9.43E−11 | 56.4 | 0.27 |
| | 47 | Fc = 4-1 | 9.01E+05 | 8.60E−05 | 9.55E−11 | 74.1 | 0.332 |
| | 17 | Fc = 4-1 | 6.25E+05 | 6.00E−05 | 9.60E−11 | 186.7 | 0.735 |
| | 53 | Fc = 4-1 | 8.08E+05 | 8.26E−05 | 1.02E−10 | 76.3 | 0.803 |
| | 43 | Fc = 4-1 | 8.35E+05 | 8.64E−05 | 1.03E−10 | 59.2 | 0.153 |
| | 5 | Fc = 2-1 | 1.05E+06 | 1.17E−04 | 1.11E−10 | 28.1 | 0.203 |
| | 46 | Fc = 4-1 | 8.23E+05 | 1.06E−04 | 1.29E−10 | 82.7 | 0.261 |
| | 9 | Fc = 3-1 | 8.71E+05 | 1.27E−04 | 1.46E−10 | 49.5 | 1.76 |
| | 55 | Fc = 4-1 | 4.82E+06 | 7.16E−04 | 1.48E−10 | 48.1 | 1.59 |
| | 51 | Fc = 2-1 | 1.15E+06 | 2.79E−04 | 2.42E−10 | 59.2 | 1.86 |
| | 1 | Fc = 2-1 | 5.10E+04 | 5.15E−04 | 1.01E−08 | 18.4 | 0.2 |

Negative controls

| | | | | | | |
|---|---|---|---|---|---|---|
| SASA | Fc = 4-1 | 7.84E+03 | 9.25E−07 | 1.18E−10 | 19.7 | 0.755 |
| SASA | Fc = 4-1 | 7.84E+03 | 9.25E−07 | 1.18E−10 | 19.7 | 0.755 |
| SASA | Fc = 2-1 | 1.52E+04 | 4.71E−06 | 3.10E−10 | 21.7 | 0.7 |
| SASA | Fc = 2-1 | 1.52E+04 | 4.71E−06 | 3.10E−10 | 21.7 | 0.7 |
| SASA | Fc = 3-1 | 2.70E+04 | 2.81E−05 | 1.04E−09 | 7.4 | 0.783 |

TABLE 10
Multi-cycle affinity measurement of binding between hTL1A
and humanized antibodies using SPR.

| Ligand | Analyte | kₐ (1/Ms) | k_{d} (1/s) | K_{D} (M) | Rmax | Chi² | U-value |
|---|---|---|---|---|---|---|---|
| AS12816 | TL1A | 8.19E+05 | 2.29E− | 2.79E− | 202.6 | 1.51 | 5 |
| AS12819 | TL1A | 9.46E+05 | 1.37E− | 1.45E− | 164.9 | 1.08 | 9 |
| AS12823 | TL1A | 1.11E+06 | <1.00E− | 9.03E− | 175.4 | 1.34 | 12 |
| AS12824 | TL1A | 1.07E+06 | <1.00E− | 9.39E− | 272.9 | 1.6 | 95 |
| BM-SH | TL1A | 9.61E+05 | 5.03E− | 5.24E− | 201.3 | 2.07 | 3 |
| Chimeric | TL1A | 1.26E+06 | 1.15E− | 9.09E− | 261.6 | 1.02 | 7 |

### Example 9

A binding competition assay using surface plasmon resonance (SPR) is performed to evaluate whether a test anti-TL1A antibody binds to the same region on TL1A as any anti-TL1A antibody described herein. In this example, the reference antibody comprises the heavy chain CDRs of SEQ ID NOs: 6-8 and the light chain CDRs of SEQ ID NOs: 14-16.

The reference antibody is directly immobilized via amine coupling onto a carboxymethylated dextran sensor chip surface (CMS) using a Biacore 2000 or 3000 instrument. Recombinant soluble human TL1A or murine TL1A diluted to 10 nMin 8.1 mM Na₂HPO₄, 1.47 mM KF₂PO₄, PH 7.2, 237 mM NaCl, 2.7 mM KCl, 3.4 mMEDTA and 0.01% tween 20 (PBS-NET) is injected for about 1 minute at a flow rate of 10 RI/minute to achieve binding levels on the immobilized antibody of at least 100 response units (RU). The reference antibody is then injected at 30 nM for 5 minutes in order to saturate all of the potential binding sites on the TL1A. A repeat injection of the reference antibody is performed to confirm this saturation. Next, the test antibody in PBS-NET or PBS-NET alone as a control is injected at 30 nM for 5 minutes. If the test antibody binds to the TL1A saturated with the first antibody, this indicates that the test antibody binds to a non-competing region on TL1A as compared to the reference antibody. If the test antibody did not bind to the saturated TL1A, this indicates that the two antibodies bind to the same region or compete with binding to TL1A. This strategy may be repeated with the test antibody immobilized and the reference antibody injected after the test antibody was bound with TL1A. Each cycle may be repeated. At the end of each cycle, the immobilized antibody surface is regenerated either by a 30-second pulse of 3M MgCl₂ or by 0.1% TFA followed by two consecutive 15-second pulses of PBS-NET. All injections are performed at 25° C. at a collection rate of 10 Hz. All sensorgrams are double referenced by using both a control surface and buffer injections.

### Example 10

Another binding competition assay using SPR is performed to evaluate whether a test anti-TL1A antibody binds to the same region on TL1A as any anti-TL1A antibody described herein. In this example, the reference antibody comprises the heavy chain CDRs of SEQ ID NOs: 6-8 and the light chain CDRs of SEQ ID NOs: 14-16.

The reference antibody is immobilized to the SPR chip via amine coupled at three or four different densities across the array. The TL1A protein is injected in an increasing concentration series to estimate kinetic parameters and the appropriate concentration for injections during the competition binning experiment. Once the optimal antigen concentration for the binning experiment is determined, regeneration conditions (typically a brief low pH injection) are evaluated to establish the optimal conditions for regeneration between cycles of the binning assay.

Binning is performed using the Pre-Mix approach, where a moderate concentration of TL1A is injected over the array, either by itself, or pre-complexed to the test antibody at saturating antibody concentrations (e.g., 30-50 μg/mL). The assay may be performed such that the test antibody is immobilized and the reference antibody is pre-complexed to the TL1A. Clones that bind to unique regions from the immobilized antibody provide an increase in signal, while competitive clones will decrease the antigen binding signal. The competition assay is run so that all clones are tested as both ligands and analytes.

Various embodiments are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments known to the applicant at this time of filing the application has been presented and is intended for the purposes of illustration and description. The present description is not intended to be exhaustive nor limited to the precise form disclosed and many modifications and variations are possible in the light of the above teachings. The embodiments described serve to explain principles and practical applications, and to enable others skilled in the art to utilize the various embodiments, optionally with various modifications, as are suited to the particular use contemplated. Therefore, it is intended that the disclosure not be limited to the particular embodiments disclosed.

While particular embodiments have been shown and described, it will be obvious to those skilled in the art that, based upon the teachings herein, changes and modifications may be made without departing from this disclosure and its broader aspects and, therefore, the appended claims are to encompass within their scope all such changes and modifications as are within the true spirit and scope of this disclosure. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A nucleic acid encoding an antibody or antigen-binding fragment that specifically binds to a tumor necrosis factor-like protein 1A (TL1A) polypeptide, wherein said antibody or antigen-binding fragment comprises:
a) a heavy chain comprising a heavy chain complementarity determining region 1 (HCDR1), a heavy chain complementarity determining region 2 (HCDR2), and a heavy chain complementarity determining region 3 (HCDR3), wherein
i) the HCDR1 comprises a first amino acid sequence of DTYMH of SEQ ID NO. 6;
ii) the HCDR2 comprises a second amino acid sequence of PASGH of SEQ ID NO. 7; and
iii) the HCDR3 comprises a third amino acid sequence of SGGLPD of SEQ ID NO. 8; and
b) a light chain comprising a light chain complementarity determining region I (LCDRI), a light chain complementarity determining region 2 (LCDR2), and a light chain complementarity determining region 3 (LCDR3), wherein
i) the LCDR1 comprises a fourth amino acid sequence of ASSSVSYMY of SEQ ID NO.14;
ii) the LCDR2 comprises a fifth amino acid sequence of ATSNLAS of SEQ ID NO. 15; and
iii) the LCDR3 comprises a sixth amino acid sequence of GNPRT of SEQ ID NO. 16.

2. The nucleic acid of claim 1, wherein the antibody or antigen-binding fragment is a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof.

3. The nucleic acid of claim 1, wherein the antibody or antigen-binding fragment is a humanized antibody.

4. The nucleic acid of claim 1, wherein the antibody or antigen-binding fragment is an immunoglobulin G (IgG).

5. The nucleic acid of claim 4, wherein the IgG comprises an IgG1.

6. The nucleic acid of claim 4, wherein the IgG comprises an IgG2.

7. A vector comprising a nucleic acid of claim 1that encodes an antibody or antigen-binding fragment, wherein the antibody or antigen-binding fragment comprises:
a) a heavy chain comprising a heavy chain complementarity determining region 1 (HCDR1), a heavy chain complementarity determining region 2 (HCDR2), and a heavy chain complementarity determining region 3 (HCDR3), wherein:
i) the HCDR1 comprises a first amino acid sequence of DTYMH of SEQ ID NO. 6;
ii) the HCDR2 comprises a second amino acid sequence of PASGH of SEQ ID NO. 7; and
iii) the HCDR3 comprises a third amino acid sequence of SGGLPD of SEQ ID NO. 8; and
b) a light chain comprising a light chain complementarity determining region I (LCDRI), a light chain complementarity determining region 2 (LCDR2), and a light chain complementarity determining region 3 (LCDR3), wherein:
i) the LCDR1 comprises a fourth amino acid sequence of ASSSVSYMY of SEQ ID NO.14;
ii) the LCDR2 comprises a fifth amino acid sequence of ATSNLAS of SEQ ID NO. 15; and
iii) the LCDR3 comprises a sixth amino acid sequence of GNPRT of SEQ ID NO. 16.

8. The vector of claim 7, wherein the vector is an expression vector.

9. The vector of claim 7, wherein the vector is a recombinant vector.

10. A host cell comprising the nucleic acid of claim 1.

11. A host cell comprising the vector of claim 7.

12. A method of treating inflammatory bowel disease (IBD) in a subject comprising administering to the subject an antibody or antigen-binding fragment, wherein the antibody or antigen-binding fragment comprises:
a) a heavy chain comprising a heavy chain complementarity determining region 1 (HCDR1), a heavy chain complementarity determining region 2 (HCDR2), and a heavy chain complementarity determining region 3 (HCDR3), wherein:
i) the HCDR1 comprises a first amino acid sequence of DTYMH of SEQ ID NO. 6;
ii) the HCDR2 comprises a second amino acid sequence of PASGH of SEQ ID NO. 7; and
iii) the HCDR3 comprises a third amino acid sequence of SGGLPD of SEQ ID NO. 8; and
b) a light chain comprising a light chain complementarity determining region I (LCDRI), a light chain complementarity determining region 2 (LCDR2), and a light chain complementarity determining region 3 (LCDR3), wherein:
i) the LCDR1 comprises a fourth amino acid sequence of ASSSVSYMY of SEQ ID NO.14;
ii) the LCDR2 comprises a fifth amino acid sequence of ATSNLAS of SEQ ID NO. 15; and
iii) the LCDR3 comprises a sixth amino acid sequence of GNPRT of SEQ ID NO. 16.

13. The method of claim 12, wherein IBD is Crohn's Disease, ulcerative colitis, medically refractory ulcerative colitis.

14. The method of claim 12, wherein the subject overexpresses TL1A.

15. The method of claim 12, wherein the antibody or antigen-binding fragment is a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof.

16. The method of claim 12, wherein the antibody or antigen-binding fragment is a humanized antibody.

17. The method of claim 12, wherein the antibody or antigen-binding fragment is an immunoglobulin G (IgG).

18. The method of claim 17, wherein the IgG comprises an IgG1.

19. The method of claim 17, wherein the IgG comprises an IgG2.


SEQUENCE LISTING (49 sequences)

SEQ ID NO: 1 - DNA, 405 bp, synthetic construct
  misc_feature 1..405: note = Description of Artificial Sequence: Synthetic polynucleotide
  source 1..405: mol_type = other DNA
atgaaatgcagctgggttatcttcttcctgatggcagtggttacaggggtcaattcagaggttcagctgcagcagtctggggcagaacttgtgaagccaggggcctcagtcaagttgtcctgcacagcttctggcttcgacattcaagacacctatatgcactgggtgaagcagaggcctgaacagggcctggagtggattggaaggattgatcctgcgagtggacatactaaatatgacccgaagttccaggtcaaggccactataacaacggacacatcctccaacacagcctacctgcagctcagcagcctgacatctgaggacactgccgtctattactgttctagatcggggggcctacctgatgtctggggcgcagggaccacggtcaccgtctcctca

SEQ ID NO: 2 - DNA, 15 bp, synthetic construct
  misc_feature 1..15: note = Description of Artificial Sequence: Synthetic oligonucleotide
  source 1..15: mol_type = other DNA
gacacctatatgcac

SEQ ID NO: 3 - DNA, 51 bp, synthetic construct
  misc_feature 1..51: note = Description of Artificial Sequence: Synthetic oligonucleotide
  source 1..51: mol_type = other DNA
aggattgatcctgcgagtggacatactaaatatgacccgaagttccaggtc

SEQ ID NO: 4 - DNA, 21 bp, synthetic construct
  misc_feature 1..21: note = Description of Artificial Sequence: Synthetic oligonucleotide
  source 1..21: mol_type = other DNA
tcggggggcctacctgatgtc

SEQ ID NO: 5 - protein, 135 aa, synthetic construct
  REGION 1..135: note = Description of Artificial Sequence: Synthetic polypeptide
MKCSWVIFFLMAVVTGVNSEVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 6 - protein, 5 aa, synthetic construct
  REGION 1..5: note = Description of Artificial Sequence: Synthetic peptide
DTYMH

SEQ ID NO: 7 - protein, 17 aa, synthetic construct
  REGION 1..17: note = Description of Artificial Sequence: Synthetic peptide
RIDPASGHTKYDPKFQV

SEQ ID NO: 8 - protein, 7 aa, synthetic construct
  REGION 1..7: note = Description of Artificial Sequence: Synthetic peptide
SGGLPDV

SEQ ID NO: 9 - DNA, 384 bp, synthetic construct
  misc_feature 1..384: note = Description of Artificial Sequence: Synthetic polynucleotide
  source 1..384: mol_type = other DNA
atggattttcaagtgcagattttcagcttcctgctaatcagtgcttcagtcataatgtccagaggacaaattgttctctcccagtctcctgcaatcctgtctgcatctccaggggagaaggtcacaatgacttgcagggccagctcaagtgtaagttacatgtactggtaccagcagaagcctggatcctcccccaaaccctggatttatgccacatccaacctggcttctggagtccctgatcgcttcagtggcagtgggtctgggacctcttactctctcacaatcagcagagtggaggctgaagatgctgccacttattactgccagcagtggagtggtaacccacggacgttcggtggaggcaccaagctggaaatcaaa

SEQ ID NO: 10 - DNA, 30 bp, synthetic construct
  misc_feature 1..30: note = Description of Artificial Sequence: Synthetic oligonucleotide
  source 1..30: mol_type = other DNA
agggccagctcaagtgtaagttacatgtac

SEQ ID NO: 11 - DNA, 21 bp, synthetic construct
  misc_feature 1..21: note = Description of Artificial Sequence: Synthetic oligonucleotide
  source 1..21: mol_type = other DNA
gccacatccaacctggcttct

SEQ ID NO: 12 - DNA, 27 bp, synthetic construct
  misc_feature 1..27: note = Description of Artificial Sequence: Synthetic oligonucleotide
  source 1..27: mol_type = other DNA
cagcagtggagtggtaacccacggacg

SEQ ID NO: 13 - protein, 128 aa, synthetic construct
  REGION 1..128: note = Description of Artificial Sequence: Synthetic polypeptide
MDFQVQIFSFLLISASVIMSRGQIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 14 - protein, 10 aa, synthetic construct
  REGION 1..10: note = Description of Artificial Sequence: Synthetic peptide
RASSSVSYMY

SEQ ID NO: 15 - protein, 7 aa, synthetic construct
  REGION 1..7: note = Description of Artificial Sequence: Synthetic peptide
ATSNLAS

SEQ ID NO: 16 - protein, 9 aa, synthetic construct
  REGION 1..9: note = Description of Artificial Sequence: Synthetic peptide
QQWSGNPRT

SEQ ID NO: 17 - DNA, 405 bp, synthetic construct
  misc_feature 1..405: note = Description of Artificial Sequence: Synthetic polynucleotide
  source 1..405: mol_type = other DNA
atgggatggagctgggtctttctcttcctcctgtcagtgactgcaggtgtccactcccaggttcacctgcagcagtctggacctgaactggtaaagcctggggcttcagtgaagttgtcctgcaaggcttctggctacaccttcacaaagtatgatataaactgggtgaggcagaggcctgaacagggacttgagtggattggatggatttttcctggagatggtagaactgactacaatgagaagttcaagggtaaggccacactgactacagacaaatcctccagcacagcctacatggaggtcagcaggctgacatctgaggactctgctgtctatttctgtgcaagatatggccccgctatggactactggggtcaaggaacctcagtcaccgtcgcctca

SEQ ID NO: 18 - DNA, 15 bp, synthetic construct
  misc_feature 1..15: note = Description of Artificial Sequence: Synthetic oligonucleotide
  source 1..15: mol_type = other DNA
aagtatgatataaac

SEQ ID NO: 19 - DNA, 51 bp, synthetic construct
  misc_feature 1..51: note = Description of Artificial Sequence: Synthetic oligonucleotide
  source 1..51: mol_type = other DNA
tggatttttcctggagatggtagaactgactacaatgagaagttcaagggt

SEQ ID NO: 20 - DNA, 20 bp, synthetic construct
  misc_feature 1..20: note = Description of Artificial Sequence: Synthetic oligonucleotide
  source 1..20: mol_type = other DNA
tatggccccgctatggacta

SEQ ID NO: 21 - protein, 135 aa, synthetic construct
  REGION 1..135: note = Description of Artificial Sequence: Synthetic polypeptide
MGWSWVFLFLLSVTAGVHSQVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 22 - protein, 5 aa, synthetic construct
  REGION 1..5: note = Description of Artificial Sequence: Synthetic peptide
KYDIN

SEQ ID NO: 23 - protein, 17 aa, synthetic construct
  REGION 1..17: note = Description of Artificial Sequence: Synthetic peptide
WIFPGDGRTDYNEKFKG

SEQ ID NO: 24 - protein, 7 aa, synthetic construct
  REGION 1..7: note = Description of Artificial Sequence: Synthetic peptide
YGPAMDY

SEQ ID NO: 25 - DNA, 393 bp, synthetic construct
  misc_feature 1..393: note = Description of Artificial Sequence: Synthetic polynucleotide
  source 1..393: mol_type = other DNA
atgaagttgcctgttaggctgttggtgctgatgttctggattcctgcttccagcagtgatgttttgatgacccaaactccactctccctgcctgtcagtcttggagatcaagcctccatctcttgcagatctagtcagaccattgtacatagtaatggagacacctatttagactggttcctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 26 - DNA, 48 bp, synthetic construct
  misc_feature 1..48: note = Description of Artificial Sequence: Synthetic oligonucleotide
  source 1..48: mol_type = other DNA
agatctagtcagaccattgtacatagtaatggagacacctatttagac

SEQ ID NO: 27 - DNA, 21 bp, synthetic construct
  misc_feature 1..21: note = Description of Artificial Sequence: Synthetic oligonucleotide
  source 1..21: mol_type = other DNA
aaagtttccaaccgattttct

SEQ ID NO: 28 - DNA, 27 bp, synthetic construct
  misc_feature 1..27: note = Description of Artificial Sequence: Synthetic oligonucleotide
  source 1..27: mol_type = other DNA
tttcaaggttcacatgttccgtacacg

SEQ ID NO: 29 - protein, 131 aa, synthetic construct
  REGION 1..131: note = Description of Artificial Sequence: Synthetic polypeptide
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 30 - protein, 16 aa, synthetic construct
  REGION 1..16: note = Description of Artificial Sequence: Synthetic peptide
RSSQTIVHSNGDTYLD

SEQ ID NO: 31 - protein, 7 aa, synthetic construct
  REGION 1..7: note = Description of Artificial Sequence: Synthetic peptide
KVSNRFS

SEQ ID NO: 32 - protein, 9 aa, synthetic construct
  REGION 1..9: note = Description of Artificial Sequence: Synthetic peptide
FQGSHVPYT

SEQ ID NO: 33 - protein, 194 aa, synthetic construct
  REGION 1..194: note = Description of Artificial Sequence: Synthetic polypeptide
QLRAQGEASVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 34 - protein, 251 aa, synthetic construct
  REGION 1..251: note = Description of Artificial Sequence: Synthetic polypeptide
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 35 - protein, 116 aa, synthetic construct
  REGION 1..116: note = Description of Artificial Sequence: Synthetic polypeptide
QVQLVQSGAEVKKPGASVKVSCKASGFDICDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 36 - protein, 116 aa, synthetic construct
  REGION 1..116: note = Description of Artificial Sequence: Synthetic polypeptide
QVQLVQSGAEVKKPGASVKLSCKASGFDICDTYMHWVRQRPGQGLEWIGRIDPASGHTKYDPKFQVRATMTTDTSTSTVYLELSSLRSEDTAVYYCARSGGLPDVWGQGTTVTVSS

SEQ ID NO: 37 - protein, 116 aa, synthetic construct
  REGION 1..116: note = Description of Artificial Sequence: Synthetic polypeptide
QVQLVQSGAEVVKPGASVKLSCKASGFDICDTYMHWVRQRPGQGLEWMGRIDPASGHTKYDPKFQVRVTMTTDTSTSTVYLELSSLRSEDTAVYYCARSGGLPDVWGQGTTVTVSS

SEQ ID NO: 38 - protein, 116 aa, synthetic construct
  REGION 1..116: note = Description of Artificial Sequence: Synthetic polypeptide
QVQLVQSGAEVKKPGASVKVSCKASGFDICDTYMHWVKQRPGQGLEWIGRIDPASGHTKYDPKFQVRATITRDTSTSTAYLELSSLRSEDTAVYYCSRSGGLPDVWGQGTTVTVSS

SEQ ID NO: 39 - protein, 116 aa, synthetic construct
  REGION 1..116: note = Description of Artificial Sequence: Synthetic polypeptide
QVQLVQSGAEVKKPGASVKVSCKASGFDICDTYMHWVKQAPGQGLEWMGRIDPASGHTKYDPKFQVRATMTTDTSTSTAYLELSSLRSEDTAVYYCSRSGGLPDVWGQGTTVTVSS

SEQ ID NO: 40 - protein, 106 aa, synthetic construct
  REGION 1..106: note = Description of Artificial Sequence: Synthetic polypeptide
EIVLTQSPGTLSASPGERATMSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 41 - protein, 106 aa, synthetic construct
  REGION 1..106: note = Description of Artificial Sequence: Synthetic polypeptide
EIVLTQSPGTLSLSPGERATMSCRASSSVSYMYWYQQKPGQAPRPWIYATSNLASGIPDRFSGSGSGTDFTLTISRVEPEDFAVYYCQQWSGNPRTFGGGTKLEIK

SEQ ID NO: 42 - protein, 106 aa, synthetic construct
  REGION 1..106: note = Description of Artificial Sequence: Synthetic polypeptide
EIVLTQSPGTLSLSPGERVTMSCRASSSVSYMYWYQQKPGQAPRPWIYATSNLASGVPDRFSGSGSGTDFTLTISRVEPEDFAVYYCQQWSGNPRTFGGGTKVEIK

SEQ ID NO: 43 - protein, 106 aa, synthetic construct
  REGION 1..106: note = Description of Artificial Sequence: Synthetic polypeptide
EIVLTQSPGTLSASPGERVTLSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 44 - protein, 106 aa, synthetic construct
  REGION 1..106: note = Description of Artificial Sequence: Synthetic polypeptide
EIVLTQSPGTLSASPGERVTMSCRASSSVSYMYWYQQKPGQAPRLLIYATSNLASGVPDRFSGSGSGTDFTLTISRVEPEDFAVYYCQQWSGNPRTFGGGTKLEIK

SEQ ID NO: 45 - protein, 6 aa, synthetic construct
  REGION 1..6: note = Description of Artificial Sequence: Synthetic 6xHis tag
HHHHHH

SEQ ID NO: 46 - protein, 135 aa, synthetic construct; identical to SEQ ID NO: 21
  REGION 1..135: note = Description of Artificial Sequence: Synthetic polypeptide

SEQ ID NO: 47 - protein, 135 aa, synthetic construct; identical to SEQ ID NO: 5
  REGION 1..135: note = Description of Artificial Sequence: Synthetic polypeptide

SEQ ID NO: 48 - protein, 115 aa, synthetic construct
  REGION 1..115: note = Description of Artificial Sequence: Synthetic polypeptide
SSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 49 - protein, 109 aa, synthetic construct
  REGION 1..109: note = Description of Artificial Sequence: Synthetic polypeptide
SRGQIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK